(19) United States
(12) Patent Application Publication
(10) Pub. No.: US 2006/0058314 A1
(43) Pub. Date: Mar. 16, 2006

(54) Oxazolidinone derivatives and their use as antibacterial agents

(75) Inventors: Michael Barry Gravestock, Waltham, MA (US); Hoan Khai Huynh, Waltham, MA (US); Neil James Hales, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/539,482

(22) PCT Filed: Dec. 15, 2003
(86) PCT No.: PCT/GB03/05448

§ 371 Date: Jun. 17, 2005

(30) Foreign Application Priority Data
Dec. 19, 2002 (GB) 0229526.9

(51) Int. Cl. C07D 417/02; C07D 413/02; A61K 31/506; A61K 31/501; A61K 31/497; A61K 31/433; A61K 31/427

(52) U.S. Cl. 514/252.05; 514/255.05; 514/256; 514/362; 514/365; 544/238; 544/333; 544/405; 546/271.4; 548/134; 548/202

(57) ABSTRACT

A compound of the formula (I), or a pharmaceutically-acceptable salt, or in-vivo hydrolysable ester thereof: (I) wherein in (I) C is for example wherein A and B are independently selected from i) ii) and m is 1 or 2; R2b and R6b, R2a and R6a, R3a and R5a, are for example selected from H, F, OMe and Me; R2b′ and R6b′, R2a′ and R6a′, R3a′, R5a′ are for example selected from H, OMe and Me; R1a is for example optionally substituted (1-10C)alkyl; R1b is for example selected from NR5C(═W)R4, a), or b) wherein HET-1 is for example isoxazolyl and HET-2 is for example triazolyl or tetrazolyl. Methods for making compounds of the formula (I), compositions containing them and their use as antibacterial agents are also described.

The present invention relates to antibiotic compounds and in particular to antibiotic compounds containing substituted oxazolidinone and/or isoxazoline rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (NMSA), methicillin resistant coagulase negative staphylococci (MACNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A. Gregory et al in J. Med. Chem. 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds containing new, more potent, pharmacophores.

We have discovered a class of bi-aryl antibiotic compounds containing two substituted oxazolidinone and/or isoxazoline rings which has useful activity against Gram-positive pathogens including MRSA and MRCNS and, in particular, against various strains exhibiting resistance to vancomycin and/or linezolid and against E. faecium strains resistant to both aminoglycosides and clinically used β-lactams, but also to fastidious Gram negative strains such as H. influenzae, M. catarrhalis, mycoplasma spp. and chlamydial strains. The compounds of the invention contain two groups capable of acting as pharmacophores. The two groups may independently bind at pharmacophore binding sites where the sites may be similar or different, where the similar or different sites may be occupied simultaneously or not simultaneously within a single organism, or where the relative importance of different binding modes to the similar or different sites may vary between two organisms of different genus. Alternatively one of the groups may bind at a pharmacophore binding site whilst the other group fulfills a different role in the mechanism of action.

Accordingly the present invention provides a compound of the formula (I), or a pharmaceutically-acceptable salt, or an in-vivo-hydrolysable ester thereof, 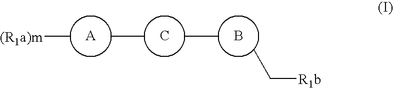
wherein in (I) C is a biaryl group C′-C″ 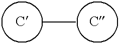
wherein C″ is an heteroaryl- or aryl-group selected from benzen-1,4-diyl, thien-2,5-diyl, and pyrid-2,5-diyl as shown in C″-1 to C″-3 below 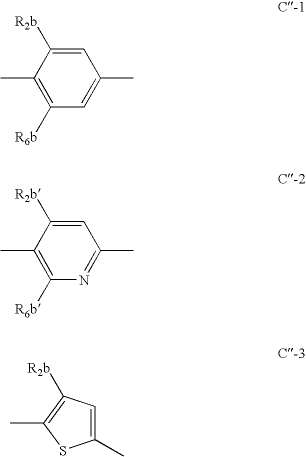
and C′ is an heteroaryl-group selected from pyridazin-3,6-diyl, pyrazin-2,5-diyl, pyrimidin-2,5-diyl (in either orientation), 1,3,4-thiadiazol-2,5-diyl, thiazol-2,5-diyl (in either orientation), and thiazol-2,4-diyl (in either orientation) as shown in C′-1 to C′-9 below: 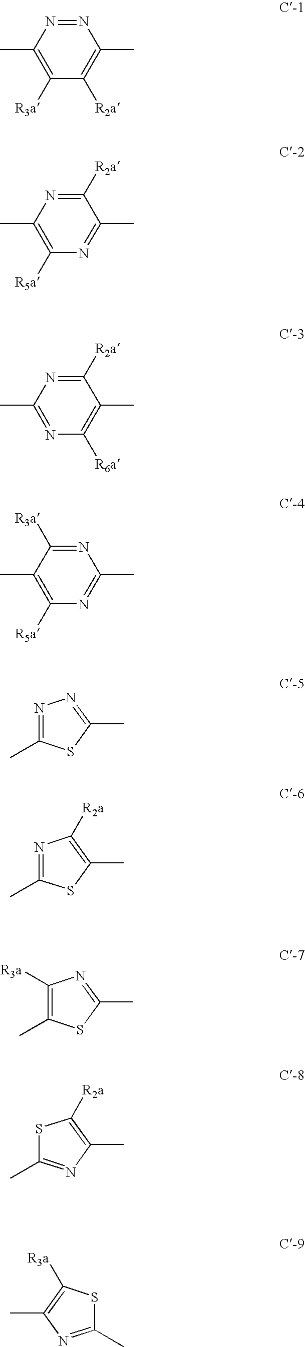
such that the central fragment C is represented by any one of the groups D to AD below: 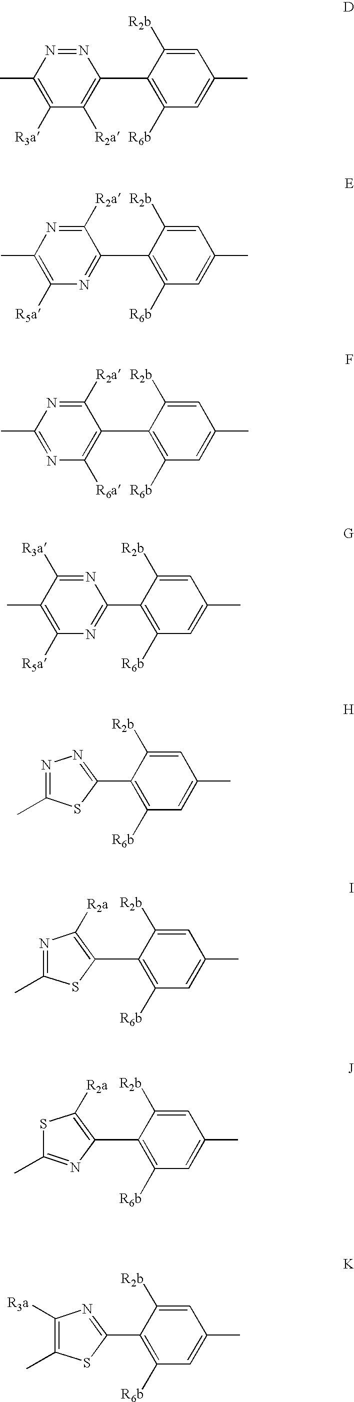 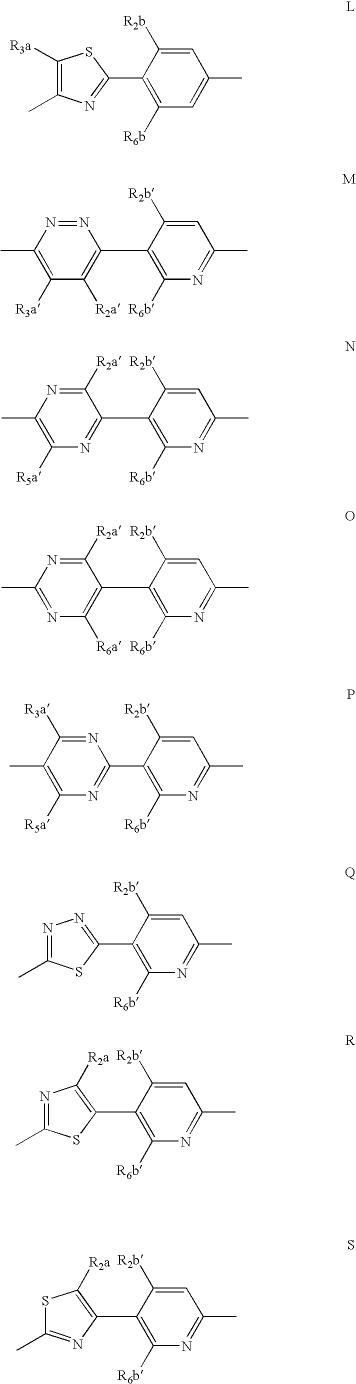 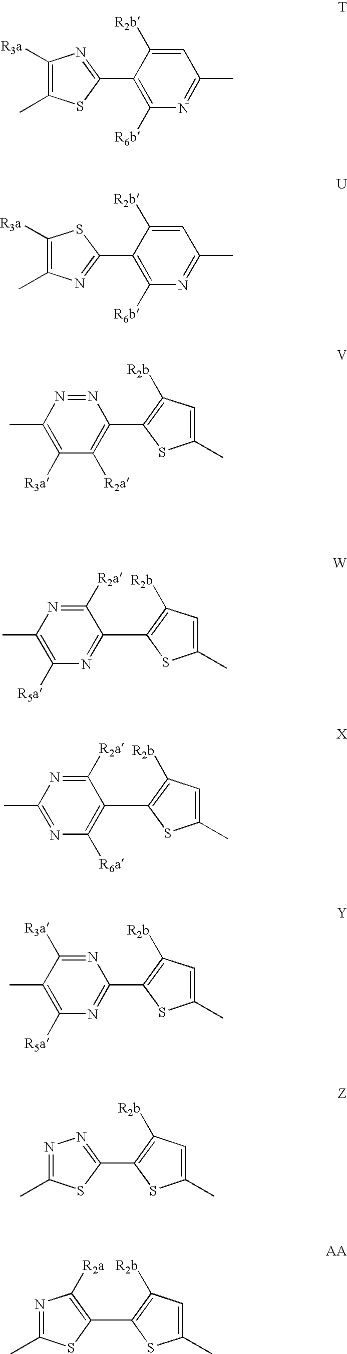 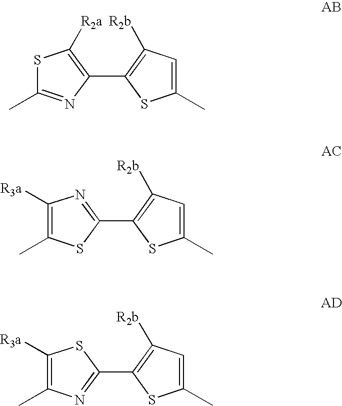
wherein the groups D to AD are attached to rings A and B in the orientation shown [(A-C′) and (C″B)];
wherein A and B are independently selected from 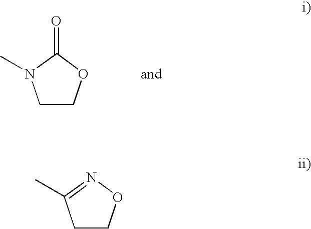
wherein A is linked as shown in (I) via the 3-position to ring C′ of group C and independently substituted in the 4 and 5 positions as shown in (I) by one or more substituents —(R₁a)m;
and wherein B is linked as shown in (I) via the 3-position to ring C″ of group C and independently substituted in the 5 position as shown in (I) by substituent —CH₂—R₁b;
R₂b and R₆b are independently selected from H, F, Cl, OMe, Me, Et and CF₃;
R₂b′ and R₆b′ are independently selected from H, OMe, Me, Et and CF₃;
R₂a is independently selected from H, Br, F, Cl, OMe, SMe; Me, Et and CF₃;
R₂a′ and R₆a′ are independently selected from H, OMe, SMe; Me, Et and CF₃;
R₃a is independently selected from H, (1-4C)alkyl, Br, P, Cl, OH, (1-4C)alkoxy, —S(O)ₙ(1-4C)alkyl (wherein n=0, 1, or 2), amino, (1-4C)alkylcarbonylamino-, nitro, cyano, —CHO, —CO(1-4C) alkyl, —CONH₂ and —CONH(1-4C)alkyl;
R₃a′ and R₅a′ are independently selected from H, (1-4C)alkyl, OH, (1-4C)alkoxy, (1-4C)alkylthio, amino, (1-4C)alkylcarbonylamino-, nitro, cyano, —CHO, —CO(1-4C)alkyl, —CONH₂ and —CONH(1-4C)alkyl;
wherein one of R₃a, R₃a′, R₅a′ taken together with a substituent R₁a at position 4 of ring A and rings A and C′ may form a 5-7 membered ring;
wherein any (1-4C)alkyl group may be optionally substituted with F, OH, (1-4C)alkoxy, —S(O)ₙ(1-4C)alkyl (wherein n=0, 1, or 2) or cyano;
wherein when ring C′ is a diazine ring (D, E, F, G, M, N, O, P, V, W, X, Y) one of the ring nitrogens may optionally be oxidised to an N-oxide;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₁a3: (1-10C)alkyl
{optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N—(1-4C)alkyl-N—(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; (═NORv) wherein Rv is as hereinbefore defined, (1-4C)alkylS(O)ₚNH—, (1-4C)alkylS(O)ₚ—((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1—O—, AR2—O—, AR3—O—, AR1—S(O)_{q}—, AR2—S(O)_{q}—, AR3—S(O)_{q}—, AR1—NH—, AR2—NH—, AR3—NH— (p is 1 or 2 and q is 0, 1 or 2), and also AR2a, AR2b, AR3a and AR3b versions of AR2 and AR3 containing groups, and additionally (1-6C)alkanoyloxy(1-4C)alkoxy, carboxy(1-4C)alkoxy, halo(1-4C)alkoxy, dihalo(1-4C)alkoxy, trihalo(1-4C)alkoxy, morpholino-ethoxy, (N′-methyl)piperazino-ethoxy, 2-, 3-, or 4-pyridyl(1-6C)alkoxy, N-methyl(imidazo-2 or 3-yl)(1-4C)alkoxy, imidazo-1-yl(1-6C)alkoxy}; wherein any (1-4C)alkyl, (1-4C)alkanoyl and (3-6C)cycloalkyl group present in any substituent on R₁a3 may itself be substituted by one or two groups selected from cyano, hydroxy, halo, amino, (1-4C)alkylamino and di(1-4C)alkylamino, provided that such a substituent is not on a carbon adjacent to a heteroatom if present;
R₁a4: R₁₄C(O)O(1-6C)alkyl wherein R₁₄ is AR1, AR2, AR2a, AR2b, (1-4C)alkylamino, or (1-10C)alkyl (optionally substituted as defined for (R₁a3), or alternatively R₁₄ is benzyloxy-(1-4C)alkyl, naphthylmethyl, (1-4C)alkoxy-(1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy, (1-4C)alkoxy-(1-4C)alkoxy-(1-4C)alkoxy, (1-4C)alkoxy-(1-4C)alkoxy, imidazo-1-yl(1-6C)alkyoxy(1-4C)alkyl, morpholino-ethoxy(1-4C)alkyl, (N′-methyl)piperazino-ethoxy(1-4C)alkyl, 2-, 3-, or 4-pyridyl(1-6C)alkyloxy(1-4C)alkyl, 2-, 3-, or 4-pyridyl(1-6C)alkylamino(1-4C)alkyl, 2-, 3-, or 4-pyridyl(1-6C)alkylsulfonyl(1-4C)alkyl, or N-methyl(imidazo-2 or 3-yl)(1-4C)alkyloxy(1-4C)alkyl;
R₁a5: F, Cl, hydroxy, mercapto, (1-4C)alkylS(O)p—(p=0,1 or 2), —OSO₂(1-4C)alkyl, —NR₁₂R₁₃, —O(1-4C)alkanoyl, —OR₁a3;
m is 0, 1 or 2;
wherein two substituents R₁a at the 4 or 5 position of ring A taken together may form a 5 to 7 membered spiro ring;
wherein two substituents R₁a at the 4 and 5 positions of ring A taken together may form a 5 to 7 membered fused ring;
R₁b is independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 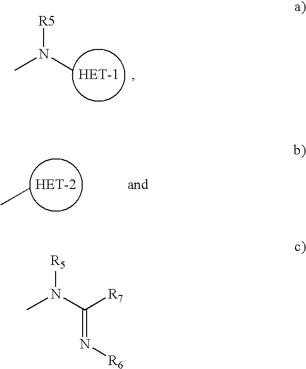
   wherein W is O or S;
R₄ is hydrogen, amino, (1-8C)alkyl, —NHR₁₂, —N(R₁₂)(R₁₃), —OR₁₂ or —SR₁₂, (2-4C)alkenyl, —(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2, and additionally (2-6C)alkyl (substituted by 1, 2 or 3 substituents independently selected from methyl, chloro, bromo, fluoro, methoxy, methylthio, azido and cyano), and methyl (substituted by 1, 2 or 3 substituents independently selected from methyl, chloro, bromo, fluoro, methoxy, methylthio, hydroxy, benzyloxy, ethynyl, (1-4C)alkoxycarbonyl, azido and cyano);
R₅ is hydrogen, (3-6C)cycloalkyl, phenyloxycarbonyl, tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;
HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quarternised) by (1-4C)alkyl; RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro, and additionally (1-4C)alkoxycarbonyl; or
(RTa2) (1-4C)alkylamino, di-(1-4C)alkylamino, and (2-4C)alkenylamino; or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl; or RT is selected from the group
RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, —(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring);
R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may additionally be taken together with the nitrogen atom to which they are attached to form an unsubstituted or substituted pyrrolidinyl, piperidinyl or morpholinyl ring, which ring may be optionally substituted by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl;
AR1 is an optionally substituted phenyl or optionally substituted naphthyl;
AR2 is an optionally substituted 5- or 6-membered, fully unsaturated (i.e. with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;
AR3 is an optionally substituted 8-, 9- or 10-membered, fully unsaturated (i.e. with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e. with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system; AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring;
wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by substituents selected independently from hydroxy, trifluoromethyl, (1-4C)alkyl S(O)q—(q is 0, 1 or 2), (1-4C)alkoxy,
(1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)q—(q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl;
Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)q—(q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

In this specification, HET-1A and HET-1B are fully unsaturated ring systems.

In this specification, HET-2A may be a fully or partially unsaturated heterocyclic ring, provided there is some degree of unsaturation in the ring.

Particular examples of 5-membered heteroaryl rings containing 2 to 4 heteroatoms independently selected from N, O and S (with no O—O, O—S or S—S bonds) are pyrazole, imidazole, 1,2,3-triazole, 1,2,4-triazole, oxazole, isoxazole, thiazole, 1,2,3-oxadiazole, 1,2,4-oxadiazole, 1,2,5-oxadiazole, 1,3,4oxadiazole, isothiazole, 1,2,5-thiadiazole, 1,2,4-thiadiazole and 1,2,3-thiadiazole.

Particular examples of 6-membered heteroaryl ring systems containing up to three nitrogen heteroatoms are pyrimidine, pyridazine, pyrazine, 1,2,3-triazine, 1,2,4-triazine and 1,3,5-triazine.

Particular examples of N-linked 5-membered, fully or partially unsaturated heterocyclic rings, containing either (i) 1 to 3 further nitrogen heteroatoms or (ii) a further heteroatom selected from O and S together with an optional further nitrogen heteroatom include, for example, pyrazole, imidazole, 1,2,3-triazole (preferably 1,2,3-triazol-1-yl), 1,2,4-triazole (preferably 1,2,4-triazol-1-yl) and tetrazole (preferably tetrazol-2-yl) and furazan.

Particular examples of N-linked 6-membered di-hydro-heteroaryl rings containing up to three nitrogen heteroatoms in total (including the linking heteroatom) include di-hydro versions of pyrimidine, pyridazine, pyrazine, 1,2,3-triazine, 1,2,4-triazine, 1,3,5-triazine and pyridine.

Particular examples of halogen-substituted alkyl substituents in HET-1 and HET-2 are monofluoromethyl, difluoromethyl and trifluoromethyl.

A particular example of R₈ as a halogen-substituted alkyl group is trifluoromethyl.

In this specification the term ‘alkyl’ includes straight chained and branched structures. For example, (1-4C)alkyl includes propyl and isopropyl. However, references to individual alkyl groups such as “propyl” are specific for the straight chained version only, and references to individual branched chain alkyl groups such as “isopropyl” are specific for the branched chain version only. A similar convention applies to other radicals, for example halo(1-4C)alkyl includes 1-bromoethyl and 2-bromoethyl.

In this specification, the terms ‘alkenyl’ and ‘cycloalkenyl’ include all positional and geometrical isomers.

In this specification, the term ‘aryl’ is an unsubstituted carbocyclic aromatic group, in particular phenyl, 1- and 2-naphthyl.

In this specification, where it is stated that a ring may be linked via an sp² carbon atom it is to be understood that the ring is linked via one of the carbon atoms in a C═C double bond.

For the avoidance of doubt, reference to a carbon atom in HET1 or HET2 being substituted by an oxo or thioxo group means replacement of a CH₂ by C═O or C═S respectively.

Within this specification composite terms are used to describe groups comprising more that one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propxyethoxymethyl.

It will be understood that where a group is defined such that is optionally substituted by more than one substituent, then substitution is such that chemically stable compounds are formed. For example, a trifluoromethyl group may be allowed but not a trihydroxymethyl group. This convention is applied wherever optional substituents are defined.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl and (1-5C)alkyl include methyl, ethyl, propyl, isopropyl and t-butyl; examples of (1-6C)alkyl include methyl, ethyl, propyl, isopropyl, t-butyl, pentyl and hexyl; examples of (1-10C)alkyl include methyl, ethyl, propyl, isopropyl, pentyl, hexyl, heptyl, octyl and nonyl; examples of (1-4C)alkanoylamino-(1-4C)alkyl include formamidomethyl, acetamidomethyl and acetamidoethyl; examples of hydroxy(1-4C)alkyl and hydroxy(1-6C)alkyl include hydroxymethyl, 1-hydroxyethyl, 2-hydroxyethyl and 3-hydroxypropyl; examples of hydroxy(2-4C)alkyl include 1-hydroxyethyl, 2-hydroxyethyl, 2-hydroxypropyl, 3-hydroxypropyl, 1-hydroxyisopropyl and 2-hydroxyisopropyl; examples of dihydroxy(1-4C)alkyl include 1,2-dihydroxyethyl, 1,2-dihydroxypropyl, 2,3-dihydroxypropyl and 1,3-dihydroxypropyl; examples of trihydroxy(1-4C)alkyl include 1,2,3-trihydroxypropyl; examples of (1-4C)alkoxycarbonyl include methoxycarbonyl, ethoxycarbonyl and propoxycarbonyl; examples of (1-5C)alkoxycarbonyl include methoxycarbonyl, ethoxycarbonyl, propoxycarbonyl and pentoxycarbonyl; examples of 2-((1-4C)alkoxycarbonyl)ethenyl include 2-(methoxycarbonyl)ethenyl and 2-(ethoxycarbonyl)ethenyl; examples of 2-cyano-2-((1-4C)alkyl)ethenyl include 2-cyano-2-methylethenyl and 2-cyano-2-ethylethenyl; examples of 2-nitro-2-((1-4C)alkyl)ethenyl include 2-nitro-2-methylethenyl and 2-nitro-2-ethylethenyl; examples of 2-((1-4C)alkylaminocarbonyl)ethenyl include 2-(methylaminocarbonyl)ethenyl and 2-(ethylaminocarbonyl)ethenyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkylsulfonyl include methylsulfonyl and ethylsulfonyl; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkoxy-(1-4C)alkoxy-(1-4C)alkoxy include 2-(methoxymethoxy)ethoxy, 2-(2-methoxyethoxy)ethoxy; 3-(2-methoxyethoxy)propoxy and 2-(2-ethoxyethoxy)ethoxy; examples of (1-4C)alkoxy-(1-4C)alkoxy-(1-4C)alkoxy-(1-4C)alkoxy-(1-4C)alkoxy-(1-4C)alkoxy include methoxyethoxyethoxyethoxyethoxyethoxy; examples of (1-4C)alkoxy-(1-4C)alkoxy-(1-4C)alkoxy-(1-4C)alkoxy-(1-4C)alkoxy include methoxyethoxyethoxyethoxyethoxy; examples of (1-4C)alkoxy-(1-4C)alkoxy-(1-4C)alkoxy-(1-4C)alkoxy include methoxyethoxyethoxyethoxy; examples of (1-4C)alkylS(O)₂amino include methylsulfonylamino and ethylsulfonylamino; examples of (1-4C)alkanoylamino and (1-6C)alkanoylamino include formamido, acetamido and propionylamino; examples of (1-4C)alkoxycarbonylamino include methoxycarbonylamino and ethoxycarbonylamino; examples of N-(1-4C)alkyl-N-(1-6C)alkanoylamino include N-methylacetamido, N-ethylacetamido and N-methylpropionamido; examples of (1-4C)alkylS(O)ₚNH— wherein p is 1 or 2 include methylsulfinylamino, methylsulfonylamino, ethylsulfinylamino and ethylsulfonylamino; examples of (1-4C)alkylS(O)ₚ((1-4C)alkyl)N— wherein p is 1 or 2 include methylsulfinylmethylamino, methylsulfonylmethylamino, 2-(ethylsulfinyl)ethylamino and 2-(ethylsulfonyl)ethylamino; examples of fluoro(1-4C)alkylS(O)ₚNH— wherein p is 1 or 2 include trifluoromethylsulfinylamino and trifluoromethylsulfonylamino; examples of fluoro(1-4C)alkylS(O)ₚ((1-4C)alkyl)NH— wherein p is 1 or 2 include tifluoromethylsulfinylmethylamino and trifluoromethylsulfonylmethylamino examples of (1-4C)alkoxy(hydroxy)phosphoryl include methoxy(hydroxy)phosphoryl and ethoxy(hydroxy)phosphoryl; examples of di-(1-4C)alkoxyphosphoryl include di-methoxyphosphoryl, di-ethoxyphosphoryl and ethoxy(methoxy)phosphoryl; examples of (1-4C)alkylS(O)_{q}— wherein q is 0, 1 or 2 include methylthio, ethylthio, methylsulfinyl, ethylsulfinyl, methylsulfonyl and ethylsulfonyl; examples of phenylS(O)_{q} and naphthylS(O)_{q}— wherein q is 0, 1 or 2 are phenylthio, phenylsulfinyl, phenylsulfonyl and naphthylthio, naphthylsulfinyl and naphthylsulfonyl respectively; examples of benzyloxy-(1-4C)alkyl include benzyloxymethyl and benzyloxyethyl; examples of a (3-4C)alkylene chain are trimethylene or tetramethylene; examples of hydroxy-(2-6C)alkoxy include 2-hydroxyethoxy and 3-hydroxypropoxy; examples of (1-6C)alkoxy-(1-6C)alkyl and (1-4C)alkoxy(1-4C)alkyl include methoxymethyl, ethoxymethyl and propoxyethyl; examples of di(1-4C)alkoxy(1-4C)alkyl include dimethoxymethyl, diethoxymethyl, 1,2-dimethoxyethyl, 1,2-diethoxyethyl, 2,3-dimethoxypropyl and 1,3-dimethoxypropyl; examples of (1-4C)alkoxy-hydroxy(1-4C)alkyl include 3-methoxy-2-hydroxypropyl, 3-hydroxy-2-methoxypropyl, 3-ethoxy-2-hydroxypropyl and 2-methoxy-2-hydroxyethyl; examples of halomethoxy(1-4C)alkyl include chloromethoxymethyl, chloromethoxyethyl, chloromethoxypropyl, chloromethoxybutyl, fluoromethoxymethyl, fluoromethoxyethyl, fluoromethoxypropyl and fluoromethoxybutyl; examples of difluoromethoxy(1-4C)alkyl include difluoromethoxymethyl, difluoromethoxyethyl and difluoromethoxypropyl; examples of dihalomethoxy(1-4C)alkyl include difluoromethoxy(1-4C)alkyl; examples of trifluoromethoxy(1-4C)alkyl include trifluoromethoxymethyl, trifluoromethoxyethyl and trifluoromethoxypropyl; examples of trihalomethoxy(1-4C)alkyl include trifluoromethoxy(1-4C)alkyl; examples of halomethoxy include chloromethoxy, chloromethoxypropyl, and fluoromethoxymethyl; examples of dihalomethoxy include difluoromethoxy; examples of trihalomethoxy include trifluoromethoxy; examples of (1-4C)alkylamino-(2-6C)alkoxy include 2-methylaminoethoxy and 2-ethylaminoethoxy; examples of di-(1-4C)alkylamino-(2-6C)alkoxy include 2-dimethylaminoethoxy and 2-diethylaminoethoxy; examples of -(1-8C)alkylaryl include benzyl and phenethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of hydroxyimino(1-4C)alkyl include hydroxyiminomethyl, 2-(hydroxyimino)ethyl and 1-(hydroxyimino)ethyl; examples of (1-4C)alkoxyimino-(1-4C)alkyl include methoxyiminomethyl, ethoxyiminomethyl, 1-(methoxyimino)ethyl and 2-(methoxyimino)ethyl; examples of halo groups include fluoro, chloro and bromo; examples of halo(1-4C)alkyl include, halomethyl, 1-haloethyl, 2-haloethyl, and 3-halopropyl; examples of dihalo(1-4C)alkyl include difluoromethyl and dichloromethyl; examples of trihalo(1-4C)alkyl include trifluoromethyl; examples of nitro(1-4C)alkyl include nitromethyl, 1-nitroethyl, 2-nitroethyl and 3-nitropropyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanesulfonamido include methanesulfonamido and ethanesulfonamido; examples of (1-4C)alkylaminosulfonyl include methylaminosulfonyl and ethylaminosulfonyl; and examples of di-(1-4C)alkylaminosulfonyl include dimethylaminosulfonyl, diethylaminosulfonyl and N-methyl-N-ethylaminosulfonyl; examples of (1-4C)alkanesulfonyloxy include methylsulfonyloxy, ethylsulfonyloxy and propylsulfonyloxy; examples of (1-4C)alkanoyloxy include acetoxy, propanoyloxy; examples of (1-6C)alkanoyloxy include acetoxy, propanoyloxy and tert-butanoyloxy; examples of (1-6C)alkanoyloxy(1-4C)alkoxy include acetoxymethoxy, propanoyloxyethoxy and tert-butylcarbonyloxymethoxy; examples of carboxy(1-4C)alkoxy include carboxymethoxy, carboxyethoxy and carboxypropoxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl; examples of (3-8C)cycloalkyl include cyclopropyl, cyclobutyl, cyclopentyl and cyclohexyl; examples of (4-7C)cycloalkyl include cyclobutyl, cyclopentyl and cyclohexyl; examples of (3-6C)cycloalkenyl include cyclopropenyl, cyclobutenyl, cyclopentenyl and cyclohexenyl; examples of di(N-(1-4C)alkyl)aminomethylimino include dimethylaminomethylimino and diethylaminomethylimino; examples of (1-4C)alkyl-S(O)q-hydroxy(1-4C)alkyl where q is 0, 1 or 2 include 3-(methylthio)-2-hydroxypropyl, 2-(methylthio)-3-hydroxypropyl, 3-(methylsulfinyl)-2-hydroxypropyl and 3-(methylsulfonyl)-2-hydroxypropyl; examples of cyano-(hydroxy)(1-4C)alkyl include 2-cyano-3-hydroxypropyl, 3-cyano-2-hydroxypropyl. Examples of morpholino-ethoxy(1-4C)alkyl and (N′-methyl)piperazino-ethoxy(1-4C)alkyl are illustrated by: 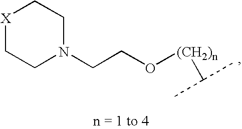
X is O or N.
Examples of 2-, 3-, or 4-pyridyl(1-6C)alkyloxy(1-4C)alkyl are illustrated by 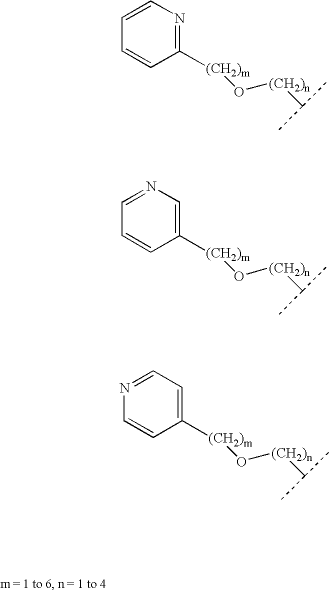
Examples of 2-, 3-, or 4-pyridyl(1-4C)alkyloxy(1-4C)alkyl are as illustrated above for 2-, 3-, or 4-pyridyl(1-6C)alkyloxy(1-4C)alkyl but wherein m=1 to 4. Examples of 2-, 3-, or 4-pyridyl(1-6C)alkylamino(1-4C)alkyl, are analogous to the alkyloxy compounds above, with NH replacing the O; similarly, examples of 2-, 3-, or 4-pyridyl(1-6C)alkylsulfonyl(1-4C)alkyl are compounds as shown above with SO₂ replacing the O. Examples of N-methyl(imidazo-2 or 3-yl)(1-4C)alkyloxy(1-4C)alkyl are illustrated by 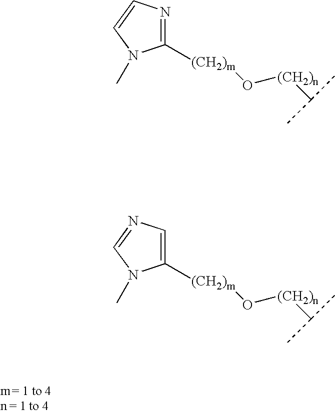
Examples of imidazo-1-yl(1-6C)alkyoxy(1-4C)alkyl are illustrated by

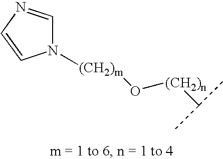

Examples of 5- and 6-membered ring acetals and methyl and phenyl derivatives thereof are 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl, 1,3-dioxan-2-yl, 2-phenyl-1,3-dioxolan-4-yl and 2-(4-methylphenyl)-1,3-dioxolan-4yl.

Particular values for AR2 include, for example, for those AR2 containing one heteroatom, furan, pyrrole, thiophene; for those AR2 containing one to four N atoms, pyrazole, imidazole, pyridine, pyrimidine, pyrazine, pyridazine, 1,2,3- & 1,2,4triazole and tetrazole; for those AR2 containing one N and one O atom, oxazole, isoxazole and oxazine; or those AR2 containing one N and one S atom, thiazole and isothiazole; or those AR2 containing two N atoms and one S atom, 1,2,4- and 1,3,4-thiadiazole.

Particular examples of AR2a include, for example, dihydropyrrole (especially 2,5-dihydropyrrol-4-yl) and tetrahydropyridine (especially 1,2,5,6-tetrahydropyrid-4-yl).

Particular examples of AR2b include, for example, tetrahydrofuran, pyrrolidine, morpholine (preferably morpholino), thiomorpholine (preferably thiomorpholino), piperazine (preferably piperazino), imidazoline and piperidine, 1,3-dioxolan-4-yl, 1,3-dioxan-4-yl, 1,3-dioxan-5-yl and 1,4-dioxan-2-yl. Further particular examples are 5- and 6-membered ring acetals as hereinbefore defined.

Particular values for AR3 include, for example, bicyclic benzo-fused systems containing a 5- or 6-membered heteroaryl ring containing one nitrogen atom and optionally 1-3 further heteroatoms chosen from oxygen, sulfur and nitrogen. Specific examples of such ring systems include, for example, indole, benzofuran, benzothiophene, benzimidazole, benzothiazole, benzisothiazole, benzoxazole, benzisoxazole, quinoline, quinoxaline, quinazoline, phthalazine and cinnoline.

Other particular examples of AR3 include 5/5-, 5/6 and 6/6 bicyclic ring systems containing heteroatoms in both of the rings. Specific examples of such ring systems include, for example, purine and naphthyridine.

Further particular examples of AR3 include bicyclic heteroaryl ring systems with at least one bridgehead nitrogen and optionally a further 1-3 heteroatoms chosen from oxygen, sulfur and nitrogen. Specific examples of such ring systems include, for example, 3H-pyrrolo[1,2-a]pyrrole, pyrrolo[2,1-b]thiazole, 1H-imidazo[1,2-a]pyrrole, 1H-imidazo[1,2-a]imidazole, 1H,3H-pyrrolo[1,2-c]oxazole, 1H-imidazo[1,5-a]pyrrole, pyrrolo[1,2-b]isoxazole, imidazo[5,1-bithiazole, imidazo[2,1-b]thiazole, indolizine, imidazo[1,2-a]pyridine, imidazo[1,5-a]pyridine, pyrazolo[1,5-a]pyridine, pyrrolo[1,2-blpyridazine, pyrrolo[1,2-c]pyrimidine, pyrrolo[1,2-a]pyrazine, pyrrolo[1,2-a]pyrimidine, pyrido[2,1-c]-s-triazole, s-triazole[1,5-a]pyridine, imidazo[1,2-c]pyrimidine, imidazo[1,2-a]pyrazine, imidazo[1,2-a]pyrimidine, imidazo[1,5-a]pyrazine, imidazo[1,5-a]pyrimidine, imidazo[1,2-b]-pyridazine, s-triazolo[4,3-a]pyrimidine, imidazo[5,1-b]oxazole and imidazo[2,1-b]oxazole. Other specific examples of such ring systems include, for example, [1H]-pyrrolo[2,1-c]oxazine, [3H]-oxazolo[3,4-a]pyridine, [6H]-pyrrolo[2,1-c]oxazine and pyrido[2,1-c][1,4]oxazine. Other specific examples of 5/5-bicyclic ring systems are imidazooxazole or imidazothiazole, in particular imidazo[5,1-b]thiazole, imidazo[2,1-b]thiazole, imidazo[5,1-b]oxazole or imidazo[2,1-b]oxazole.

Particular examples of AR3a and AR3b include, for example, indoline, 1,3,4,6,9,9a-hexahydropyrido[2,1c][1,4]oxazin-8-yl, 1,2,3,5,8,8a-hexahydroimidazo[1,5a]pyridin-7-yl, 1,5,8,8a-tetrahydrooxazolo[3,4a]pyridin-7-yl, 1,5,6,7,8,8a-hexahydrooxazolo[3,4a]pyridin-7-yl, (7aS)[3H,5H]-1,7a-dihydropyrrolo[1,2c]oxazol-6-yl, (7aS)[5H]-1,2,3,7a-tetrahydropyrrolo[1,2c]imidazol-6-yl, (7aR)[3H,5H]-1,7a-dihydropyrrolo[1,2c]oxazol-6-yl, [3H,5H]-pyrrolo[1,2-c]oxazol-6-yl, [5H]-2,3-dihydropyrrolo[1,2-c]imidazol-6-yl, [3H,5H]-pyrrolo[1,2-c]thiazol-6-yl, [3H,5H]-1,7a-dihydropyrrolo[1,2-c]thiazol-6-yl, [5H]-pyrrolo[1,2-c]imidazol-6-yl, [1H]-3,4,8,8a-tetrahydropyrrolo[2,1-c]oxazin-7-yl, [3H]-1,5,8,8a-tetrahydrooxazolo-[3,4-a]pyrid-7-yl, [3H]-5,8-dihydroxazolo[3,4-a]pyrid-7-yl and 5,8-dihydroimidazo-[1,5-a]pyrid-7-yl.

Particular values for AR4 include, for example, pyrrolo[a]quinoline, 2,3-pyrroloisoquinoline, pyrrolo[a]isoquinoline, 1H-pyrrolo[1,2-a]benzimidazole, 9H-imidazo[1,2-a]indole, 5H-imidazo[2,1-a]isoindole, 1H-imidazo[3,4-a]indole, imidazo[1,2-a]quinoline, imidazo[2,1-alisoquinoline, imidazo[1,5-a]quinoline and imidazo[5,1-a]isoquinoline.

The nomenclature used is that found in, for example, “Heterocyclic Compounds” (Systems with bridgehead nitrogen), W. L. Mosby (Interscience Publishers Inc., New York), 1961, Parts 1 and 2.

Where optional substituents are listed such substitution is preferably not geminal disubstitution unless stated otherwise. If not stated elsewhere, suitable optional substituents for a particular group are those as stated for similar groups herein.

Preferable optional substituents on Ar2b as 1,3-dioxolan-4-yl, 1,3-dioxan-4-yl, 1,3-dioxan-5-yl or 1,4-dioxan-2-yl are mono- or disubstitution by substituents independently selected from (1-4C)alkyl (including geminal disubstitution), (1-4C)alkoxy, (1-4C)alkylthio, acetamido, (1-4C)alkanoyl, cyano, trifluoromethyl and phenyl].

Preferable optional substituents on CY1 & CY2 are mono- or disubstitution by substituents independently selected from (1-4C)alkyl (including geminal disubstitution), hydroxy, (1-4C)alkoxy, (1-4C)alkylthio, acetamido, (1-4C)alkanoyl, cyano, and trifluoromethyl.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Suitable pro-drugs for pyridine or triazole derivatives include acyloxymethyl pyridinium or triazolium salts e.g. halides; for example a pro-drug such as: 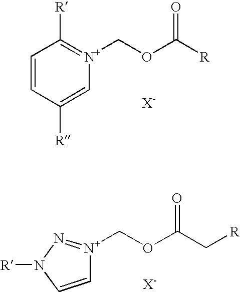
(Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (104C)alkyl or H. Further suitable prodrugs are carbonate and carbamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4(1-4C)piperazino-(1-4C)alkyl, piperazino-(1-4C)alkyl and morpholino-(1-4C)alkyl.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

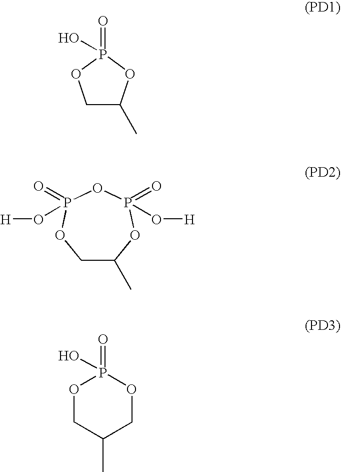

Esters of compounds of formula (I) wherein the HO— function/s in (PD1), (PD2) and (PD3) are protected by (1-4C)alkyl, phenyl or benzyl are useful intermediates for the preparation of such pro-drugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

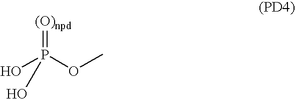

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD1) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

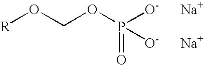

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Where pharmaceutically-acceptable salts of an in-vivo hydrolysable ester may be formed this is achieved by conventional techniques. Thus, for example, compounds containing a group of formula (PD1), (PD2), (PD3)and/or (PD4) may ionise (partially or fully) to form salts with an appropriate number of counter-ions. Thus, by way of example, if an in-vivo hydrolysable ester prodrug of a compound of invention contains two (PD4) groups, there are four HO—P-functionalities present in the overall molecule, each of which may form an appropriate salt (i.e. the overall molecule may form, for example, a mono-, di-, tri- or tetra-sodium salt).

The compounds of the present invention have a chiral centre at the C-5 position of the oxazolidinone or isoxazoline ring B. Where m>0 there may be additional chiral centres at C-4 and/or C-5 position of Ring A. The pharmaceutically active diastereomers are of the formula (Ia): 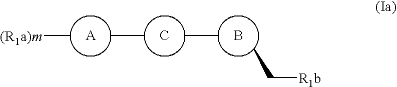
wherein the chiral centre of ring B is fixed in the orientation shown (generally the (5R) configuration, depending on the nature of R₁b, C and B) and ring B is acting as a pharmacophonic group; and wherein the substitution pattern and orientation of the chiral centre(s) at ring A may vary and may influence whether ring A also independently binds to a pharmacophore binding site.

Furthermore, some compounds of the invention may have other chiral centres. It is to be understood that the invention encompasses all such optical and diastereoisomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H. influenzae, M. catarrhalis, Mycoplasma and Chlamydia strains. The following compounds possess preferred pharmaceutical and/or physical and/or pharmacokinetic properties.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

In one aspect, an in-vivo hydrolysable ester of a compound of the formula (1) is a phosphoryl ester (as defined by formula (PD4) with npd as 1).

Compounds of the formula (I), or a pharmaceutically-acceptable salt or an in-vivo hydrolysable ester thereof, wherein C is selected from any one of groups D to AD represent separate and independent aspects of the invention.

Particularly preferred compounds of the invention comprise a compound of the invention, or a pharmaceutically-acceptable salt or an in-vivo hydrolysable ester thereof, wherein the substituents A, B, R₁a, R₁b, R₂a, R₂b, R₃a, R₂b′, R₆b, R₆b′, R₂a′, R₃a′, R₅a′ and R₆a′ and other substituents mentioned above have values disclosed hereinbefore, or any of the following values (which may be used where appropriate with any of the definitions and embodiments disclosed hereinbefore or hereinafter):

In one embodiment are provided compounds of formula (I) or a pharmaceutically-acceptable salt or an in-vivo hydrolysable ester thereof, in which group C is represented by group D.

In another embodiment are provided compounds of formula (1) or a pharmaceutically-acceptable salt or an in-vivo hydrolysable ester thereof, in which group C is represented by group E.

In another embodiment are provided compounds of formula (I) or a pharmaceutically-acceptable salt or an in-vivo hydrolysable ester thereof, in which group C is represented by group P.

In another embodiment are provided compounds of formula (1) or a pharmaceutically-acceptable salt or an in-vivo hydrolysable ester thereof, in which group C is represented by group G.

In another embodiment are provided compounds of formula a) or a pharmaceutically-acceptable salt or an in-vivo hydrolysable ester thereof, in which group C is represented by group H.

In another embodiment are provided compounds of formula (I) or a pharmaceutically-acceptable salt or an in-vivo hydrolysable ester thereof, in which group C is represented by group I.

In another embodiment are provided compounds of formula (I) or a pharmaceutically-acceptable salt or an in-vivo hydrolysable ester thereof, in which group C is represented by group J.

In another embodiment are provided compounds of formula (I) or a pharmaceutically-acceptable salt or an in-vivo hydrolysable ester thereof, in which group C is represented by group K.

In another embodiment are provided compounds of formula (I) or a pharmaceutically-acceptable salt or an in-vivo hydrolysable ester thereof, in which group C is represented by group L.

In another embodiment are provided compounds of formula (I) or a pharmaceutically-acceptable salt or an in-vivo hydrolysable ester thereof, in which group C is represented by group M.

In another embodiment are provided compounds of formula (I) or a pharmaceutically-acceptable salt or an in-vivo hydrolysable ester thereof, in which group C is represented by group N.

In another embodiment are provided compounds of formula (I) or a pharmaceutically-acceptable salt or an in-vivo hydrolysable ester thereof, in which group C is represented by group O.

In another embodiment are provided compounds of formula (I) or a pharmaceutically-acceptable salt or an in-vivo hydrolysable ester thereof, in which group C is represented by group P.

In another embodiment are provided compounds of formula (I) or a pharmaceutically-acceptable salt or an in-vivo hydrolysable ester thereof, in which group C is represented by group Q.

In another embodiment are provided compounds of formula (I) or a pharmaceutically-acceptable salt or an in-vivo hydrolysable ester thereof, in which group C is represented by group R.

In another embodiment are provided compounds of formula (I) or a pharmaceutically-acceptable salt or an in-vivo hydrolysable ester thereof, in which group C is represented by group S.

In another embodiment are provided compounds of formula (I) or a pharmaceutically-acceptable salt or an in-vivo hydrolysable ester thereof, in which group C is represented by group T.

In another embodiment are provided compounds of formula (I) or a pharmaceutically-acceptable salt or an in-vivo hydrolysable ester thereof, in which group C is represented by group U.

In another embodiment are provided compounds of formula (I) or a pharmaceutically-acceptable salt or an in-vivo hydrolysable ester thereof, in which group C is represented by group V.

In another embodiment are provided compounds of formula (I) or a pharmaceutically-acceptable salt or an in-vivo hydrolysable ester thereof, in which group C is represented by group W.

In another embodiment are provided compounds of formula (I) or a pharmaceutically-acceptable salt or an in-vivo hydrolysable ester thereof, in which group C is represented by group X.

In another embodiment are provided compounds of formula (I) or a pharmaceutically-acceptable salt or an in-vivo hydrolysable ester thereof, in which group C is represented by group Y.

In another embodiment are provided compounds of formula (I) or a pharmaceutically-acceptable salt or an in-vivo hydrolysable ester thereof, in which group C is represented by group Z.

In another embodiment are provided compounds of formula (I) or a pharmaceutically-acceptable salt or an in-vivo hydrolysable ester thereof, in which group C is represented by group AA.

In another embodiment are provided compounds of formula (I) or a pharmaceutically-acceptable salt or an in-vivo hydrolysable ester thereof, in which group C is represented by group AB.

In another embodiment are provided compounds of formula (I) or a pharmaceutically-acceptable salt or an in-vivo hydrolysable ester thereof, in which group C is represented by group AC.

In another embodiment are provided compounds of formula (I) or a pharmaceutically-acceptable salt or an in-vivo hydrolysable ester thereof, in which group C is represented by group AD.

In another embodiment are provided compounds of formula (I) or a pharmaceutically-acceptable salt or an in-vivo hydrolysable ester thereof, in which group C is a represented by group selected from groups D, E, F, G, H, I, J, K and L as hereinbefore defined.

In another embodiment are provided compounds of formula (I) or a pharmaceutically-acceptable salt or an in-vivo hydrolysable ester thereof, in which group C is represented by a group selected from groups M, N, O, P, Q, R, S, T and U as hereinbefore defined.

In another embodiment are provided compounds of formula (I) or a pharmaceutically-acceptable salt or an in-vivo hydrolysable ester thereof, in which group C is represented by a group selected from groups V, W, X, Y, Z, AA, AB, AC and AD as hereinbefore defined.

In another embodiment are provided compounds of formula (I) or a pharmaceutically-acceptable salt or an in-vivo hydrolysable ester thereof, in which group C is represented by a group selected from groups F, H and I as hereinbefore defined. Preferably group C is group F or group I.

In one aspect both A and B are oxazoldinone rings.

In another aspect both A and B are isoxazoline rings.

In another aspect, either A or B is an oxazolidinone ring and the other is an isoxazoline ring. In this aspect, preferably A is the isoxazoline ring and B is the oxazolidinone ring.

In one aspect, R₂b and R₆b are independently H or F.

In one aspect R₂b′ and R₆b′ are both H.

When m=1, in one aspect R₁a is selected from R₁a1; in another aspect R₁a is selected from R₁a2; in a further aspect R₁a is selected from R₁a3 and in a further aspect R₁a is selected from R₁a4.

When m=2, in one aspect both groups R₁a are independently selected from the same group R₁a1 to R₁a4. In a further aspect when m=2, each R₁a is independently selected from different groups R₁a1 to R₁a4.

Conveniently, m is 1 or 2. In one aspect, preferably m is 1. In another aspect, preferably m is 2.

In one aspect, when m is 2, both substituents R₁a are attached to position 4 of ring A to form a 5-7 membered spiro-ring.

In one aspect, when m is 2, both substituents R₁a are attached to position 5 of ring A to form a 5-7 membered spiro-ring.

In another aspect, when m is 2, one substituent R₁a is attached to position 4 of ring A, and the other is attached to position 5 of ring A, such that taken together with A they form a 5-7 membered fused-ring.

In a particular aspect when m is 2, the two substituents R₁a are identical to each other, preferably selected from R₁a3 and are attached to the same position (4 or 5) of ring A such that ring A does not have a chiral centre.

Particular values for R₁a when selected from R₁a1 are AR1 and AR2, more particularly AR2.

Particular values for R₁a when selected from R₁a2 are cyano and —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl (optionally substituted on a carbon not adjacent to the nitrogen), (3-6C)cycloalkyl, (1-4C)alkanoyl, —COO(1-4C)alkyl, —S(O)n(1-4C)alkyl (wherein n=1 or 2;), —COOAR1, —CS(1-4C)alkyl and —C(═S)O(1-4C)alkyl; wherein any (1-4C)alkyl, (1-4C)alkanoyl and (3-6C)cycloalkyl is optionally substituted by cyano, hydroxy or halo]. More particular values for R₁a when selected from R₁a2 are cyano, formyl, —COO(1-4C)alkyl, —C(═O)NH₂, —(C═O)piperazine and —(C═O)morpholine.

Particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N—(1-4C)alkyl-N-(1-6C)alkanoylamino-, —C(═W)NRvRw [wherein W is O, Rv and Rw are independently H, or (1-4C)alkyl and wherein Rv and Rw taken together with the amide nitrogen to which they are attached can form a morpholine, pyrrolidine, piperidine or piperazine ring; wherein when said ring is a piperazine ring, the ring may be optionally substituted on the additional nitrogen by a group selected from (1-4C)alkyl and (1-4C)alkanoyl], (1-4C)alkylS(O)q—, (q is 0, 1 or 2), AR2, AR2—O—, AR2—NH—, and also AR2a, AR2b versions of AR2 containing groups}; wherein any (1-4C)alkyl and (1-4C)alkanoyl present in any substituent on R₁a3 may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;

More particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], carboxy, amino, (1-4C)alkylamino, di(1-4C)alkylamino, (1-4C)alkylS(O)q (preferably where q=2), AR2 and AR2b . More particular values for R₁a when selected from R₁a3 are (1-6C)alkyl substituted as hereinbefore described. Even more particular values for R₁a when selected from R₁a3 are (1-4C)alkyl substituted as hereinbefore described.

Particular values for R₁a when selected from R₁a4 are R₁₄C(O)O(1-6C)alkyl- wherein R₁₄ is selected from AR1, AR2, AR2a, AR2b and (1-10C)alkyl (optionally substituted by one or more substituents independently selected from OH and di (1-4C)alkylamino. More particular vales for R₁₄ are AR2a, AR2b and (1-6C)alkyl substituted with hydroxy. More particular values for R₁₄ are AR2a, AR2b and (1-4C)alkyl substituted with hydroxy.

In one aspect R₁a4: is R¹⁴C(O)O(1-6C)alkyl [wherein R¹⁴ is AR1, AR2, AR2a, AR2b, (1-4C)alkylamino, benzyloxy-(1-4C)alkyl or (1-10C)alkyl {optionally substituted as defined for (R₁a3)].

Particular values for R₁a when selected from R₁a5 are fluoro, chloro and hydroxy.

In a most particular aspect, R₁a is selected from (1-4C)alkyl (optionally substituted on an available carbon atom with one, two, three or more substituents independently selected from F, Cl and Br), hydroxy(2-4C)alkyl, dihydroxy(1-4C)alkyl, trihydroxy(1-4C)alkyl, (1-4C)alkoxy(1-4C)alkyl, trifluoromethoxy(1-4C)alkyl, difluoromethoxy(1-4C)alkyl, halomethoxy(1-4C)alkyl, di[(1-4C)alkoxy](1-4C)alkyl, (1-4C)alkoxy-(hydroxy)(1-4C)alkyl, (1-4C)alkyl-S(O)q-hydroxy(1-4C)alkyl (where q is 0, 1 or 2), cyano-(hydroxy)(1-4C)alkyl, morpholino-ethoxy(1-4C)alkyl, (N′-methyl)piperazino-ethoxy(1-4C)alkyl, 2-, 3-, or 4 pyridyl(1-6C)alkoxy(1-4C)alkyl, N-methyl(imidazo-2 or 3-yl)(1-4C)alkoxy(1-4C)alkyl, imidazo-1-yl(1-6C)alkoxy(1-4C)alkyl, and 5- and 6-membered ring acetals (optionally substituted with one or two substituents independently selected from methyl and phenyl (wherein the phenyl group is itself optionally substituted with one or two substituents selected from methyl, methoxy, chloro and bromo)).

In an alternative most particular aspect, R₁a is selected from (1-4C)alkyl, hydroxy(2-4C)alkyl, dihydroxy(1-4C)alkyl, trihydroxy(1-4C)alkyl, (1-4C)alkoxy(1-4C)alkyl, di[(1-4C)alkoxy](1-4C)alkyl, (1-4C)alkoxy-(hydroxy)(1-4C)alkyl, (1-4C)alkyl-S(O)q-hydroxy(1-4C)alkyl (where q is 0, 1 or 2), cyano-(hydroxy)(1-4C)alkyl, morpholino-ethoxy(1-4C)alkyl, (N′-methyl)piperazino-ethoxy(1-4C)alkyl, 2-, 3-, or 4-pyridyl(1-6C)alkoxymethyl, N-methyl(imidazo-2 or 3-yl)(1-6C) alkoxymethyl, imidazo-1-yl(1-6C)alkyl, 5- and 6-membered ring acetals (optionally substituted with one or two substituents independently selected from methyl and phenyl (wherein the phenyl group is itself optionally substituted with one or two substituents selected from methyl, methoxy, chloro and bromo)).

Further particular values for R₁a are (1-4C)alkylS(O)q—, where q is 0, 1 or 2 and wherein the (1-4C)alkyl group is optionally substituted with hydroxy.

When R₁a is selected from 2-, 3-, or 4-pyridyl(1-4C)alkyloxy(1-4C)alkyl, N-methyl(imidazo-2 or 3-yl)(1-4C)alkyloxy(1-4C)alkyl, and imidazo-1-yl(1-6C)alkyoxy(1-4C)alkyl, it is preferably selected from 2-, 3-, or 4-pyridyl(1-4C)alkyloxymethyl, N-methyl(imidazo-2 or 3-yl)(1-4C)alkyloxymethyl, and imidazo-1-yl(1-6C)alkyloxymethyl.

References hereinafter to R₁a being selected from (1-4C)alkyl include (1-4C)alkyl optionally substituted on an available carbon atom with one, two, three or more substituents independently selected from F, Cl and Br. In one embodiment, such a (1-4C)alkyl group is optionally substituted by one, two or three substituents independently selected from F, Cl and Br. In another embodiment, such a (1-4C)alkyl group is optionally substituted by one, two or three substituents independently selected from F and Cl, so that R₁a is selected from, for example, chloromethyl, fluoromethyl, difluoromethyl, trifluoromethyl, chloroethyl and fluoroethyl.

When m is 1:

in one aspect R₁a is selected from (1-4C)alkyl hydroxy(2-4C)alkyl, dihydroxy(1-4C)alkyl and trihydroxy(1-4C)alkyl;

in another aspect, R₁a is selected from (1-4C)alkoxy(1-4C)alkyl, di[(1-4C)alkoxy](1-4C)alkyl, 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl;

in a further aspect, R₁a is selected from halomethoxy(1-4C)alkyl and 2-, 3-, or 4-pyridyl(1-4C)alkyloxymethyl;

in a further aspect, R₁a is selected from trifluoromethoxy(1-4C)alkyl, difluoromethoxy(1-4C)alkyl and fluoromethoxy(1-4C)alkyl;

in a further aspect, R₁a is selected from morpholino-ethoxy(1-4C)alkyl, (N′-methyl)piperazino-ethoxy(1-4C)alkyl, 2-,3-, or 4-pyridyl(1-4C)alkyloxy(1-4C)alkyl, N-methyl(imidazo-2 or 3-yl)(1-4C)alkyloxy(1-4C)alkyl, and imidazo-1-yl(1-6C)alkyoxy(1-4C)alkyl.

When m is 1, suitably R₁a is selected from hydroxy(2-4C)alkyl and dihydroxy(1-4C)alkyl. More suitably, R₁a is selected from hydroxyethyl and 1,2-dihydroxyethyl. Preferably, when m is 1, R₁a is 1,2-dihydroxyethyl.

When m is 2:

in one aspect each R₁a is independently selected from (1-4C)alkyl, hydroxy(1-4C)alkyl, dihydroxy(1-4C)alkyl and trihydroxy(1-4C)alkyl;

in another aspect, each R₁a is independently selected from (1-4C)alkoxy(1-4C)alkyl and di[(1-4C)alkoxy](1-4C)alkyl;

in a further aspect, at least one R₁a is selected from halomethoxy(1-4C)alkyl and 2-, 3-, or 4-pyridyl(1-4C)alkyloxymethyl;

in a further aspect, at least one R₁a is selected from trifluoromethoxy(1-4C)alkyl, difluoromethoxy(1-4C)alkyl and fluoromethoxy(1-4C)alkyl;

in a further aspect, one R₁a is selected from (1-4C)alkyl, hydroxy(1-4C)alkyl, dihydroxy(1-4C)alkyl and trihydroxy(1-4C)alkyl; and the other R₁a is selected from (1-4C)alkoxy(1-4C)alkyl and di[(1-4C)alkoxy](1-4C)alkyl;

in a further aspect, one R₁a is selected from (1-4C)alkyl, hydroxy(1-4C)alkyl, dihydroxy(1-4C)alkyl and trihydroxy(1-4C)alkyl; and the other R₁a is selected from halomethoxy(1-4C)alkyl and 2-, 3-, or 4-pyridyl(1-4C)alkyloxymethyl.

When m is 2, preferably both R₁a are hydroxymethyl or both hydroxyethyl. In another aspect, when m is 2, preferably one R₁a is hydroxymethyl and the other is methoxymethyl.

In all of the embodiments, aspects and preferable values for R₁b defined hereinbefore or hereinafter, any (1-4C)alkyl group may be optionally substituted as hereinbefore defined. Particular substituents for (1-4C)alkyl groups in definitions for R₁b are one or two halogen groups, particularly geminal disubstitution (provided that such substitution is not on a carbon atom attached to an oxygen) and cyano. Examples of di-halosubstituted groups are —NHCOCF₂H and —NHCSCCl₂H.

When R₁b is —N(R₅)HET-1, R₅ is preferably hydrogen.

In one embodiment R₁b is selected from hydroxy, —NHCO(1-4C)alkyl, —NHCO(3-6C)cycloalkyl, —NHCS(1-4C)alkyl, —NHCOO(1-4C)alkyl, —NH(C═S)O(1-4C)alkyl, —OCO(1-4C)alkyl, —N(R₅)-HET-1 and HET-2.

In another embodiment R₁b is selected from —NHCO(1-4C)alkyl, —NHCO(3-6C)cycloalkyl, —NHCS(1-4C)alkyl, —N(R₅)-HET-1 and HET-2.

More preferably R₁b is selected from —NHCO(1-4C)alkyl, —NHCS(1-4C)alkyl, —N(R₅)-HET-1 and HET-2.

In one aspect, R₁b is selected from OH, —NR₅C(═W)R₄ and —OC(═O)R₄, in particular OH, —NHCOMe and —NHCOOMe.

In a further aspect, R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In a most particular aspect, R₄ is selected from the values given hereinbefore.

In one aspect R₄ is selected from hydrogen, amino, (1-8C)alkyl, —NHR₁₂, —N(R₁₂)(R₁₃), —OR₁₂ or —SR₁₂, (2-4C)alkenyl, —(1-8C)alkylaryl, mono-, di-, tri- and per-halo(1-8C)alkyl, —(CH₂)p(3-6C)cycloalkyl and —(CH₂)p(3-6C)cycloalkenyl wherein p is 0, 1 or 2;

In one embodiment R₁b is selected from hydroxy, —NHC(═W)R₄, —OC(═O)R₄, and

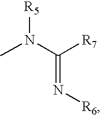

wherein W, R₅ and R₆ are as defined hereinbefore, R₄ is selected from hydrogen, amino, (1-4C)alkyl, —NH(1-4C)alkyl, —N(di-(1-4C)alkyl), —O—(1-4C)alkyl, —S(1-4C)alkyl, (2-4C)alkenyl, —(CH₂)p(3-6C)cycloalkyl and —(CH₂)p(3-6C)cycloalkenyl wherein p is 0, 1 or 2; and R₇ is selected from hydrogen, (1-8C)alkyl, —OR₁₂, —SR₁₂, amino, —NHR₁₂, —N(R₁₂)(R₁₃), (1-8C)alkylaryl and mono-, di-, tri- and per-halo(1-8C)alkyl.

In another embodiment, R₁b is selected from hydroxy, —NHC(═W)R₄, —OC(═O)R₄, 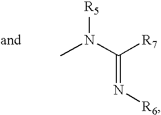
wherein W, R₄, R₅, R₆ and R₇ are as defined hereinbefore, especially wherein R₄ is (1-4C)alkyl, (1-4C)alkoxy, cycloalkyl (particularly cyclopropyl) or haloalkyl (particularly dichloromethyl).

In another embodiment, R₁b is selected from hydroxy, —NHC(═W)R₄, —OC(═O)R₄, 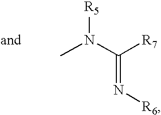
wherein W, R₄, R₅, R₆ and R₇ are as defined hereinbefore, especially wherein R₄ is (1-4C)alkyl or (1-4C)alkoxy.

Particular values for R₅ (which may be used as appropriate with any of the definitions and embodiments disclosed hereinbefore or hereinafter) are hydrogen, tert-butoxycarbonyl and benzyloxycarbonyl. More particularly, R₅ is hydrogen.

In one aspect R₁₂ and R₁₃ are independently selected from hydrogen, alkyl and aryl, or for any N(R₁₂)(R₁₃) group, R₁₂ and R₁₃ may additionally be taken together with the nitrogen atom to which they are attached to form pyrrolidinyl, piperidinyl or morpholinyl group, optionally substituted as hereinbefore described. In one aspect R₁₅ and R₁₆ are independently selected from hydrogen, phenyl and (1-4C)alkyl).

In one aspect, R₁₂ and R₁₃ are independently selected from hydrogen and methyl.

In one embodiment HET-1 and HET-2 are unsubstituted. When substituted, preferred substituents are selected from halo (particularly chloro), (1-4C)alkyl, especially methyl, mono- and di-halo methyl (wherein halo is preferably fluoro, chloro or bromo), trifluoromethyl and cyanomethyl.

Preferred are HET-1 and HET-2 as 5-membered rings, ie HET-1 as HET-1A and HET_{—}2 as HET-2A, in particular HET-1A as isoxazolyl, 1,2,5-thiadiazolyl or isothiazolyl and HET-2A as 1,2,3-triazol-1-yl or tetrazol-2-yl.

In one aspect, HET-2A as 1,2,3-triazol-1-yl is substituted, preferably by halo (particularly chloro), methyl, difluoromethyl, fluoromethyl, chloromethyl, cyanomethyl or trifluoromethyl.

In one embodiment HET-2A is selected from the structures (Za) to (Zf) below: 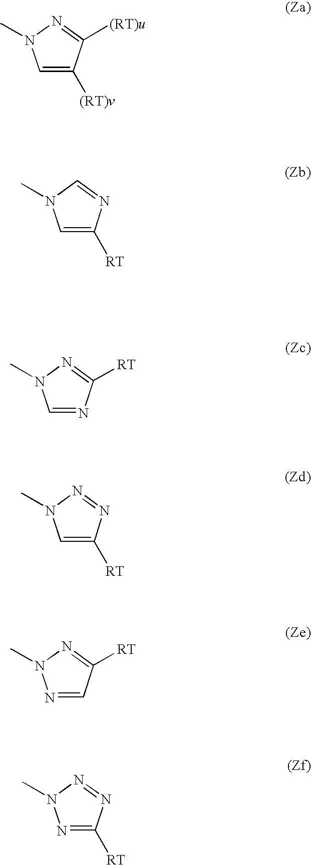
wherein u and v are independently 0 or 1 and RT is as defined in any of the embodiments or aspects defined hereinbefore or hereinafter.

In one embodiment HET-2A is selected from 1,2,3-triazole (especially 1,2,3-triazol-1-yl (Zd)), 1,2,4-triazole (especially 1,2,4-triazol-1-yl (Zc)) and tetrazole (preferably tetrazol-2-yl (Zf)) and wherein u and v are independently 0 or 1 and RT is as defined in any of the embodiments or aspects defined hereinbefore or hereinafter.

In another embodiment HET-2A is selected from 1,2,3-triazol-1-yl (Zd) and tetrazol-2-yl (Zf) and wherein u and v are independently 0 or 1 and RT is as defined in any of the embodiments or aspects defined hereinbefore or hereinafter.

In another embodiment HET-2A is 1,2,3-triazol-1-yl (Zd) and wherein u and v are independently 0 or 1 and RT is as defined in any of the embodiments or aspects defined hereinbefore or hereinafter.

In one embodiment HET-2B is a di-hydro version of pyrimidine, pyridazine, pyrazine, 1,2,3-triazine, 1,2,4-triazine, 1,3,5-triazine and pyridine and wherein RT is as defined in any of the embodiments or aspects defined hereinbefore or hereinafter.

In another embodiment HET-2B is selected from pyrimidone, pyridazinone, pyrazinone, 1,2,3-triazinone, 1,2,4-triazinone, 1,3,5-triazinone and pyridone and wherein RT is as defined in any of the embodiments or aspects defined hereinbefore or hereinafter.

In another embodiment HET-2B is selected from thiopyrimidone, thiopyridazinone, thiopyrazinone, thio-1,2,3-triazinone, thio-1,2,4-triazinone, thio-1,3,5-triazinone and thiopyridone and wherein RT is as defined in any of the embodiments or aspects defined hereinbefore or hereinafter.

In a most particular aspect, R₁b is —NH(C═W)R₄ or (Zd).

In one aspect R₁b is —NH(C═O)R₄.

In another aspect R₁b is (Zd).

When W is O, suitably R₄ is selected from methyl, ethyl, dichloromethyl and cyclopropyl.

When W is S, suitably R₄ is selected from (1-4C)alkyl (optionally substituted by 1, 2 or 3 substituents independently selected from methyl, chloro, bromo, fluoro and methoxy), —N(R₁₂)(R₁₃) and —OR₁₂. More suitably, when W is S, R₄ is selected from —NH₂, —NHMe, —OMe, —SMe and methyl.

In one aspect (RTa1)is selected from hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro.

In one aspect RT is preferably selected from a substituent from the group
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or,
(RTa2) (1-4C)alkylamino, di-(1-4C)alkylamino and (2-4C)alkenylamino;
(RTh1) a (1-4C)alkyl group which is optionally substituted by one substituent selected from hydroxy, (1-4C)alkoxy, (1-4C)alkylthio, cyano and azido; or
(RTb2) a (1-4C)alkyl group which is optionally substituted by one substituent selected from (2-4C)alkenyloxy, (3-6C)cycloalkyl and (3-6C)cycloalkenyl;
and wherein at each occurrence of an RT substituent containing an alkyl, alkenyl, alkynyl, cycloalkyl or cycloalkenyl moiety in (RTa1) or (RTa2), or (RTb1) or (RTb2) each such moiety is optionally substituted on an available carbon atom with one, two, three or more substituents independently selected from F, Cl, Br, OH and CN.

In another aspect RT is preferably selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano, and nitro; or
(RTb1) a (1-4C)alkyl group which is optionally substituted by one substituent selected from hydroxy, (1-4C)alkoxy, (1-4C)alkylthio, cyano and azido; and wherein at each occurrence of an RT substituent containing an alkyl, alkenyl, alkynyl, cycloalkyl or cycloalkenyl moiety in (RTa1) or (RTb1) each such moiety is optionally substituted on an available carbon atom with one, two, three or more substituents independently selected from F, Cl, Br, and CN.

In a further aspect RT is most preferably
(a) hydrogen; or
(b) halogen, in particular fluorine, chlorine, or bromine; or
(c) cyano; or
(d) (1-4C)alkyl, in particular methyl; or
(e) monosubstituted (1-4C)alkyl, in particular fluoromethyl, choromethyl, bromomethyl, cyanomethyl, azidomethyl, hydroxymethyl; or
(f) disubstituted (1-4C)alkyl, for example difluoromethyl, or trisubstituted (1-4C)alkyl, for example trifluoromethyl.

In a most particular aspect, RT is selected from hydrogen, halogen, cyano, (1-4C)alkyl, cyano(1-4C)alkyl, halo(1-4C)alkyl, dihalo(1-4C)alkyl, trihalo(1-4C)alkyl, amino, (1-4C)alkylamino, di-(1-4C)alkylamino, (1-4C)alkylthio, (1-4C)alkoxy, 1-4C)alkoxy(1-4C)alkyl, (2-4C)alkenyloxy, (2-4C)alkenyl, (2-4C)alkynyl, (3-6C)cycloalkyl, (3-6C)cycloalkenyl and (1-4C)alkoxycarbonyl; and wherein at each occurrence of an RT substituent containing an alkyl, alkenyl, alkynyl, cycloalkyl or cycloalkenyl moiety each such moiety is optionally substituted on an available carbon atom with one, two, three or more substituents independently selected from F, Cl, Br, OH and CN.

In one embodiment of this most particular aspect, RT is selected from hydrogen, halogen, cyano, (1-4C)alkyl, halo(1-4C)alkyl, dihalo(1-4C)alkyl and (2-4C)alkyl; suitably, RT is selected from hydrogen, chloro, bromo, fluoro, methyl, fluoromethyl, chloromethyl, bromomethyl, difluoromethyl and dichloromethyl, ethynyl and propynyl; more suitably, RT is selected from hydrogen, chloro, bromo, methyl and fluoromethyl.

In one embodiment is provided a compound of formula (I) or a pharmaceutically-acceptable salt or an in-vivo hydrolysable ester thereof, wherein group C is selected from groups D, E, P, G, H, I, J, K and L; R₂b and R₆b are independently H or F; A and B are both oxazolidinones; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (1) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and &b are independently H or F; A and B are both oxazolidinones; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both oxazolidinones; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; A and B are both isoxazolines; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or P; A and B are both isoxazolines; m=2; and R₁b is selected from —N(R₅)-HET-1 and HET-2, in particular HET-1 as isoxazolyl, 1,2,5-thiadiazolyl or isothiazolyl and HET-2 as 1,2,3-tiazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or P; either A or B is an oxazolidinone and the other is an isoxazoline; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E, F, G, H, I, J, K and L; R₂b and R₆b are independently H or F; either A or B is an oxazolidinone and the other is an isoxazoline; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or P; A and B are both oxazolidinones; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=1; R₁a is selected from R₁a3; and R₁b is selected from —N(R₅)-HET-1 and HET-2, in particular H-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or P; A and B are both isoxazolines; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₁b are independently H or F; A and B are both oxazolidinones; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both oxazolidinones; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; A and B are both isoxazolines; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups M, N, O, P, Q, R, S, T and U; R₂b and R₆b are independently H or F; either A or B is an oxazolidinone and the other is an isoxazoline; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (1) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (1) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R1a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both oxazolidinones; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; A and B are both isoxazolines; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups V, W, X, Y, Z, AA, AB, AC and AD; R₂b and R₆b are independently H or F; either A or B is an oxazolidinone and the other is an isoxazoline; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=1; R₁a is selected from R₁a1; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=1; R₁a is selected from R₁a2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆ are independently H or F; either A or B is an oxazolidinone and the other is an isoxazoline; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=1; R₁a is selected from R₁a3; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both oxazolidinones; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; A and B are both isoxazolines;
m=2; and R₁b is selected from -N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups F, H and I; R₂b and R₆b are independently H or F; either A or B is an oxazolidinone and the other is an isoxazoline; m=2; and R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl.

In all of the above definitions the preferred compounds are as shown in formula (Ia).

Particular compounds of the present invention include each individual compound described in the Examples, especially Example 1. Each Example provides an independent aspect of the invention.

Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain Patent Application Publications, the contents of the relevant process sections of which are hereby incorporated herein by reference; for example WO 94/13649; WO 98/54161; WO 99/64416; WO 99/64417; WO 00/21960; WO 01/40222.

In particular we refer to our PCT patent applications WO 99/64417 and WO 00/21960 wherein detailed guidance is given on convenient methods for preparing oxazolidinone compounds.

The skilled organic chemist will be able to use and adapt the information contained and referenced within the above references, and accompanying Examples therein and also the Examples herein, to obtain necessary starting materials, and products. For example, the skilled chemist will be able to apply the teaching herein for compounds of formula (I) in which group C is drawn from groups D to L to prepare compounds in which group C is drawn from groups M to U and groups V to AD as hereinbefore defined. Similarly, in the processes illustrated below the skilled chemist will be able to apply the relevant teaching as necessary to prepare compounds in which one or both of rings A or B is isoxazoline to prepare those compounds in which one or both rings A or B is oxazolidinone; and the skilled chemist will be able to apply the relevant teaching as necessary to prepare compounds in which one or both of rings A or B is oxazolidinone to prepare those compounds in which one or both rings A or B is isoxazoline.

Thus, the present invention also provides that the compounds of the invention and pharmaceutically-acceptable salts and in vivo hydrolysable esters thereof, can be prepared by a process (a) to (h); and thereafter if necessary:
i) removing any protecting groups;
ii) forming a pro-drug (for example an in-vivo hydrolysable ester); and/or
iii) forming a pharmaceutically-acceptable salt;
wherein said processes (a) to (h) are as follows (wherein the variables are as defined above unless otherwise stated):
(a) by modifying a substituent in, or introducing a substituent into another compound of the invention by using standard chemistry (see for example, Comprehensive Organic Functional Group Transformations (Pergamon), Katritzky, Meth-Cohn & Rees or Advanced Organic Chemistry (Wiley-Interscience), Jerry March or Houben-Weyl, Methoden der Organischen Chemie)); for example:
an acylamino group may be converted into a thioacylamino group;
an acylamino group or thioacylamino group may be converted into another acylamino or thioacylamino; heterocyclyl for instance tetrazolyl or thiazolyl, or heterocyclylamino group (optionally substituted or protected on the amino-nitrogen atom), a heterocyclyl group linked through nitrogen (optionally substituted on a carbon other than a carbon adjacent to the linking nitrogen atom), for instance an optionally 4substituted 1,2,3-triazol-1-yl group; or an amidino group; such conversions of the acylamino group taking place either directly or through through the intermediacy of one or more derivatives such as an amino group; an acyloxy group may be converted into a hydroxy group or into the groups that may be obtained from a hydroxy group (either directly or through the intermediacy of a hydroxy group);
an alkyl halide such as alkylbromide or alkyliodide may be converted into an alkyl fluoride or nitrile;
an alkyl sulfonate such as alkyl methanesulfonate may be converted into an alkyl fluoride or nitrile;
an alkylthio group such as methylthio may be converted into a methanesulfinyl or methanesulfonyl group;
an arylthio group such as phenylthio may be converted into a benzenesulfinyl or benzenesulfonyl group;
an amidino or guanidino group may be converted into a range of 2-substituted 1,3-diazoles and 1,3-diazines;
an amino group may be converted for instance into acylamino or thioacylamino for instance an acetamide (optionally substituted), alkyl- or dialkyl-amino and thence into a further range of N-alkyl-amine derivatives, sulfonylamino, sulfinylamino, amidino, guanidino, arylamino, heteroarylamino, N-linked heterocyclic for instance an optionally 4-substituted 1,2,3-triazol-1-yl group;
an aryl- or heteroaryl-halide group such as an aryl- or hetero-aryl chloride or bromide or iodide may be converted by transition metal mediated coupling, especially Pd(0) mediated coupling into a range of aryl-, heteroaryl, alkenyl, alkyl, acyl, alkylthio, or alkyl- or dialkyl-amino substituted aryl or heteroaryl groups;
an aryl- or heteroaryl-sulfonate group such as an aryl- or hetero-aryl trifluoromethanesulfonate may be converted by transition metal mediated coupling, especially Pd(0) mediated coupling into a range of aryl-, heteroaryl, alkenyl, alkynyl, acyl, alkylthio, or alkyl- or dialkyl-amino substituted aryl or heteroaryl groups;
an aryl- or heteroaryl-halide group such as an aryl- or hetero-aryl chloride or bromide or iodide may be converted by transition metal mediated coupling, especially Pd(0) mediated coupling into a range of trialkyltin, dialkylboronate, trialkoxysilyl, substituted aryl or heteroaryl groups useful as intermediates for the synthesis of compounds of the invention;
an azido group may be converted for instance into a 1,2,3-triazolyl or amine and thence by methods that are well known in the art into any of the range common amine derivatives such as acylamino for instance acetamido group;
a carboxylic acid group may be converted into trifloromethyl, hydroxymethyl, alkoxycarbonyl, aminocarbonyl optionally substituted on nitrogen, formyl, or acyl groups;
a cyano group may be converted into a tetrazole, or an imidate, an amidine, an amidrazone, an N-hydroxyamidrazone, an amide, a thioamide, an ester, or an acid and thence by methods that are well known in the art into any of the range of heterocycles derived from such nitrile derivatives;
a hydroxy group may be converted for instance into an alkoxy, cyano, azido, alkylthio, keto and oximino, fluoro, bromo, chloro, iodo, alkyl- or aryl-sulfonyloxy for instance trifluoromethanesulfonate, methanesulfonate, or tosylsulfonate, silyloxy; acylamino or thioacylamino , for instance an acetamide (optionally substituted or protected on the amido-nitrogen atom); acyloxy, for instance an acetoxy; phosphono-oxy, heterocyclylamino (optionally substituted or protected on the amino-nitrogen atom), for instance an isoxazol-3-ylamino or a 1,2,5-thiadiazol-3-ylamino; heterocyclyl linked through nitrogen (optionally substituted on a carbon other than a carbon atom adjacent to the linking nitrogen ring atom), for instance an optionally 4-substituted 1,2,3-triazol-1-yl; or amidino, for instance an 1-(N-cyanoimino)ethylamino group; such conversions of the hydroxy group taking place directly (for instance by acylation or Mitsunobu reaction) or through the intermediacy of one or more derivatives (for instance a mesylate or an azide);
a silyloxy group may be converted into a hydroxy group or into the groups that may be obtained from a hydroxy group (either directly or through the intermediacy of a hydroxy group);
a keto group may be converted into a hydroxy, thiocarbonyl, oximino, or difluoro group;
a nitro-group may be converted into an amino group and thence by methods that are well known in the art into any of the range common amine derivatives such as acylamino for instance acetamido group;
an optionally substituted aromatic or heteroaromatic ring C′ may be converted into another aromatic or heteroaromatic ring C′ by introduction of a new substituent (R2a to R6a or R2a′ or R6a′) or by refunctionalisation of an existing substituent (R2a to R6a or R2a′ or R6a′);
a heterocyclylamino group (optionally substituted or protected on the amino-nitrogen atom) may be converted into another heterocyclyl amino group (optionally substituted or protected on the amino-nitrogen atom) by refunctionalisation, for instance by protection or deprotection, of the amino-nitrogen atom, by introduction of a new ring substituent, or by refunctionalisation of an existing ring substituent;
a heterocyclyl group linked through nitrogen (optionally substituted on a carbon other than a carbon atom adjacent to the linking nitrogen ring atom) may be converted into another heterocyclyl group linked through nitrogen (optionally substituted on a carbon other than a carbon atom adjacent to the linking nitrogen ring atom) by introduction of a new ring substituent or by refunctionalisation of an existing ring substituent, for instance by modifying the 4-substituent of a 4substituted 1,2,3-triazol-1-yl group;
for instance, examples drawn from the methods for conversion of a hydroxy group into an optionally substituted triazole group are illustrated by the scheme: 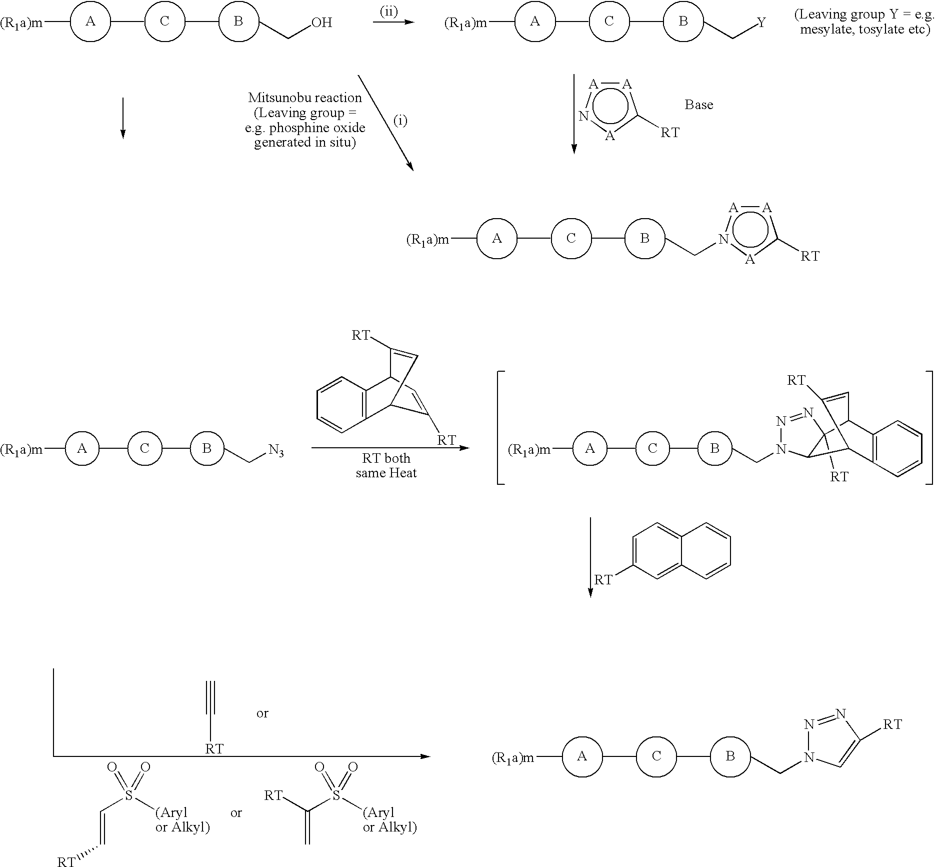
   examples drawn from the range of regioselective methods that proceed under very mild conditions are further illustrated by processes (f), (g), and (h);
examples drawn from the methods for conversion of a hydroxy group into an optionally substituted heterocyclyamino group are illustrated by the scheme: 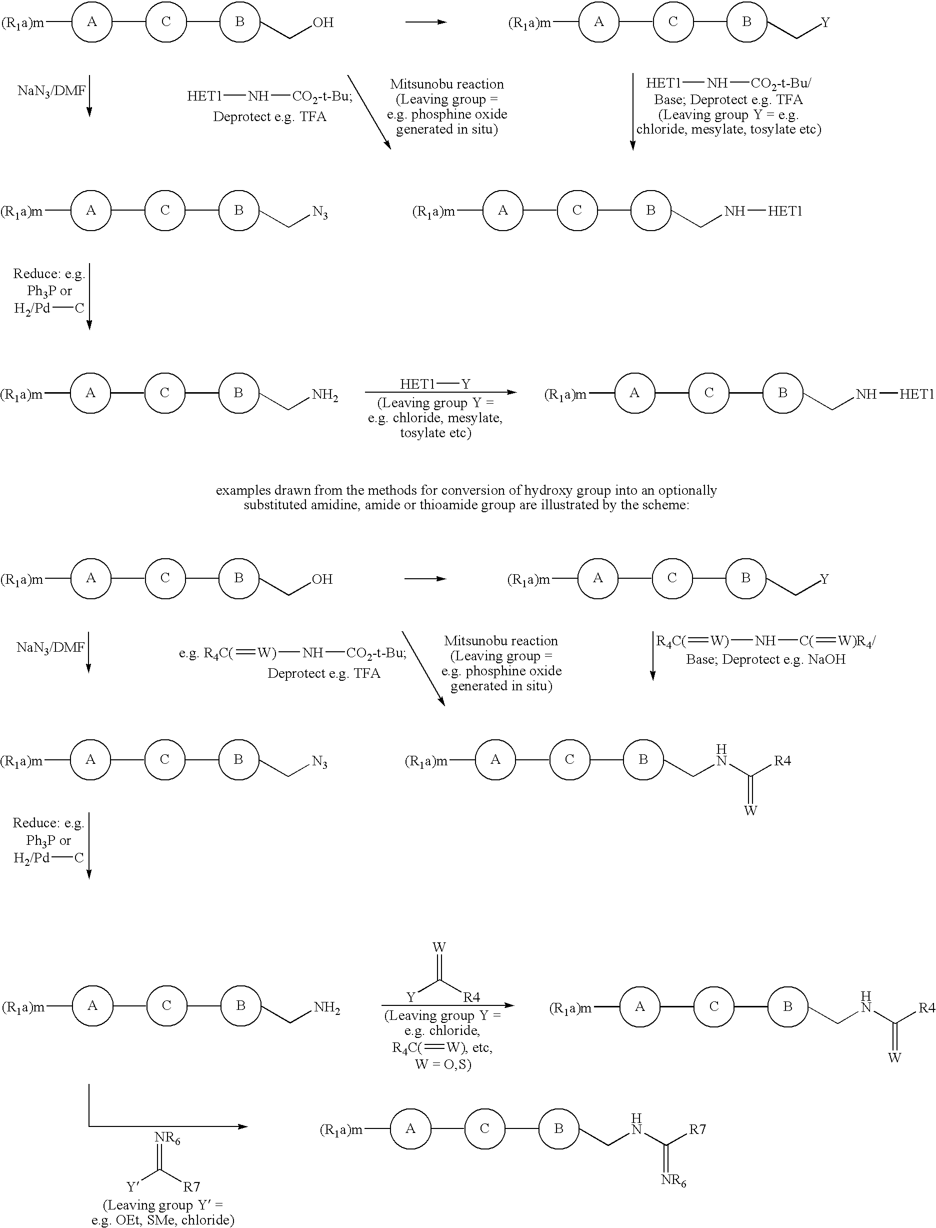
(b) by reaction of a molecule of a compound of formula (IIa) (wherein X is a leaving group useful in palladium coupling, for example boronate, trimethyl tin, trialkoxysilyl, alkanesulfonyloxy for instance trifluoromethanesulfonyloxy, iodo and bromo) with a molecule of a compound of formula (IIb) (wherein X′ is a leaving group useful in palladium coupling, for example boronate, trimethyl tin, trialkoxysilyl, alkanesulfonyloxy for instance trifluoromethanesulfonyloxy, iodo and bromo) wherein X and X′ are chosen such that an heteroaryl-aryl, or heteroaryl-heteroaryl bond replaces the heteroaryl-X and aryl-X′ (or heteroaryl-X′) bonds. Such methods are now well known, see for instance see for instance J. K. Stille, Angew Chem. Int. Ed. Eng., 1986, 25, 509-524; N. Miyaura and A Suzuki, Chem. Rev., 1995, 95, 2457-2483, D. Baranano, G. Mann, and J. F. Hartwig, Current Org. Chem., 1997, 1, 287-305, S. P. Stanforth, Tetrahedron, 54 1998, 263-303, and P. R. Parry, C. Wang, A. S. Batsanov, M. R. Bryce, and B. Tarbit, J. Org. Chem., 2002, 67, 7541-7543; 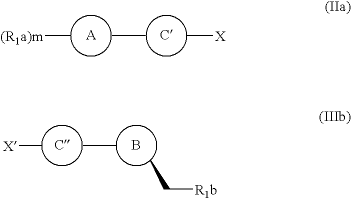
   the method can be used to prepare compounds of formula (I) or (IA) where rings A and B are the same; for instance where both rings A and B are oxazolidinones, 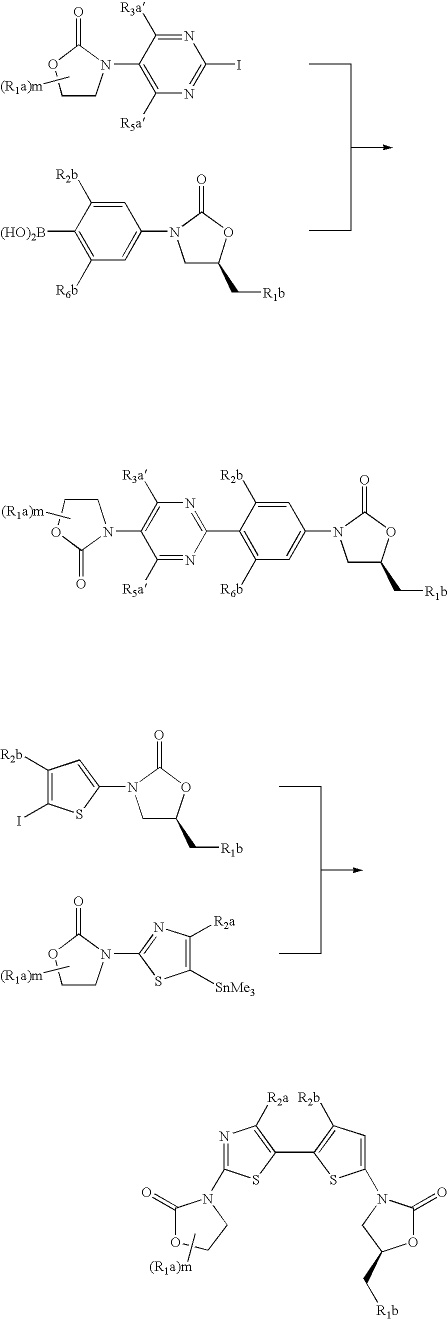 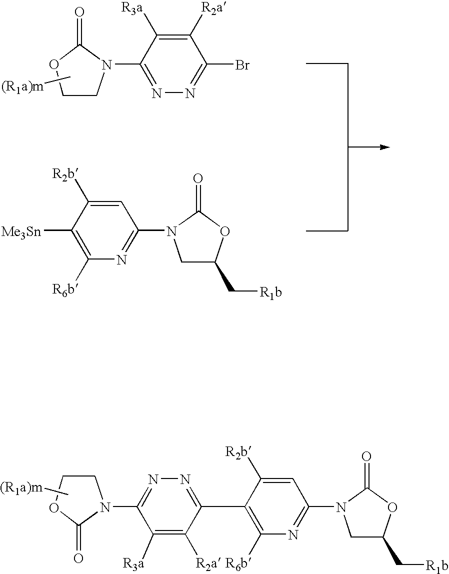
   or for instance where both rings A and B are isoxazolines: 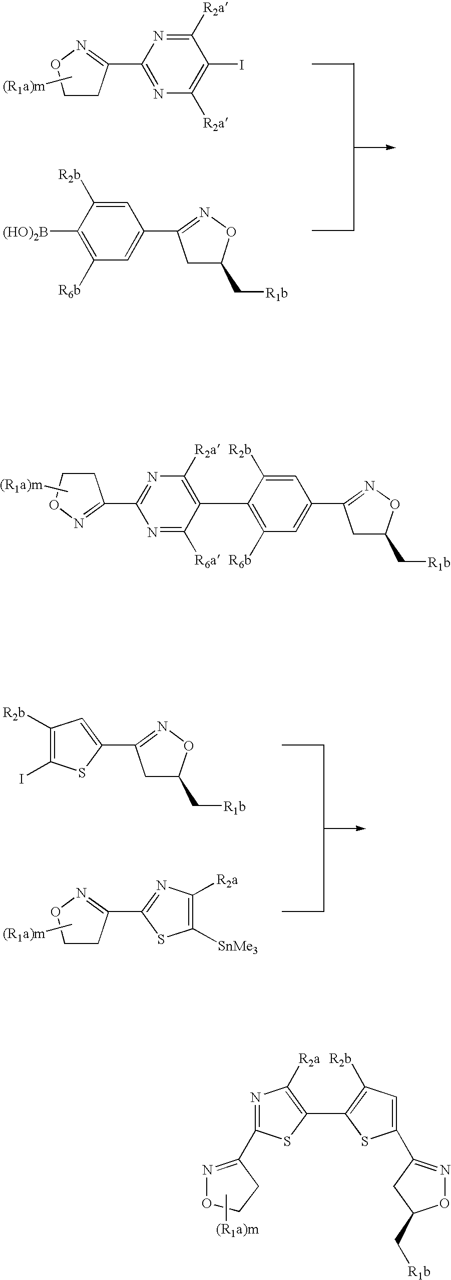 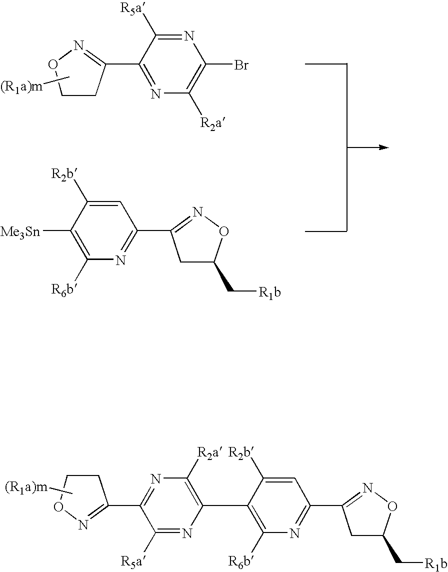
   similarly, this chemistry may be applied to the preparation of compounds of formula (I) or(IA) in which ring A and ring B are dissimilar, for instance where ring A is oxazolidinone and ring B is isoxazoline, or 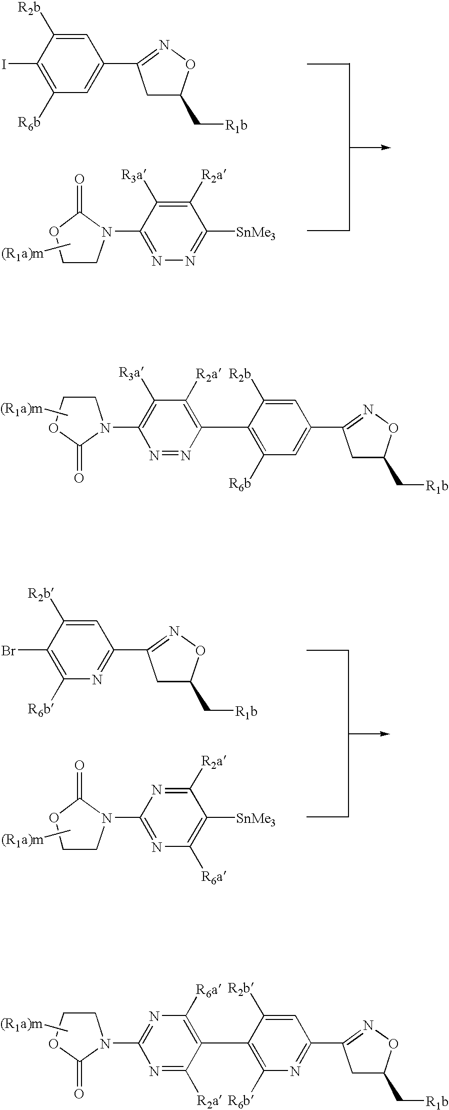
   for example in which ring A is isoxazoline and ring B is oxazolidinone; 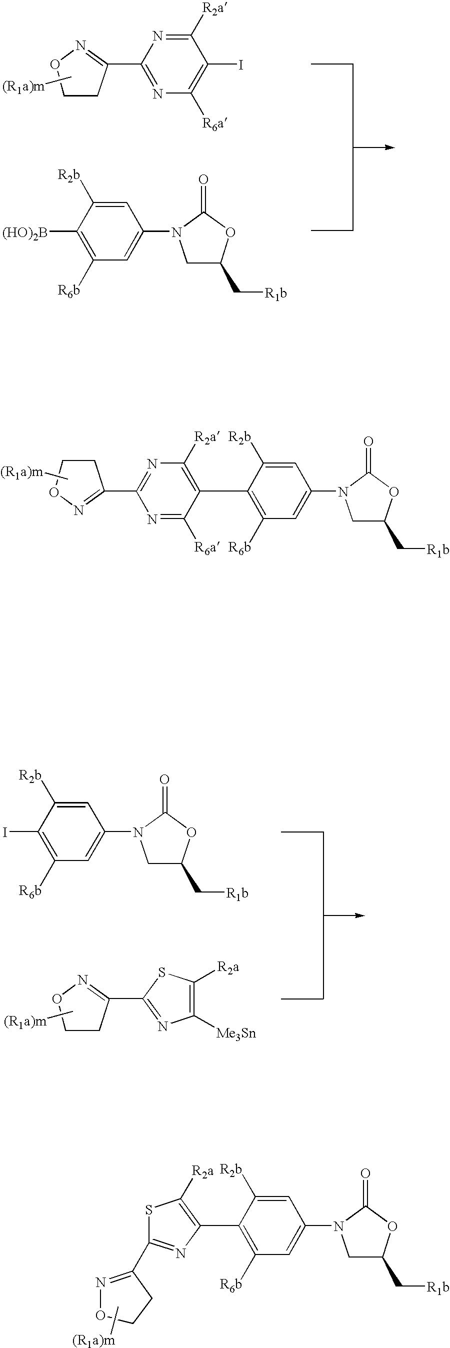 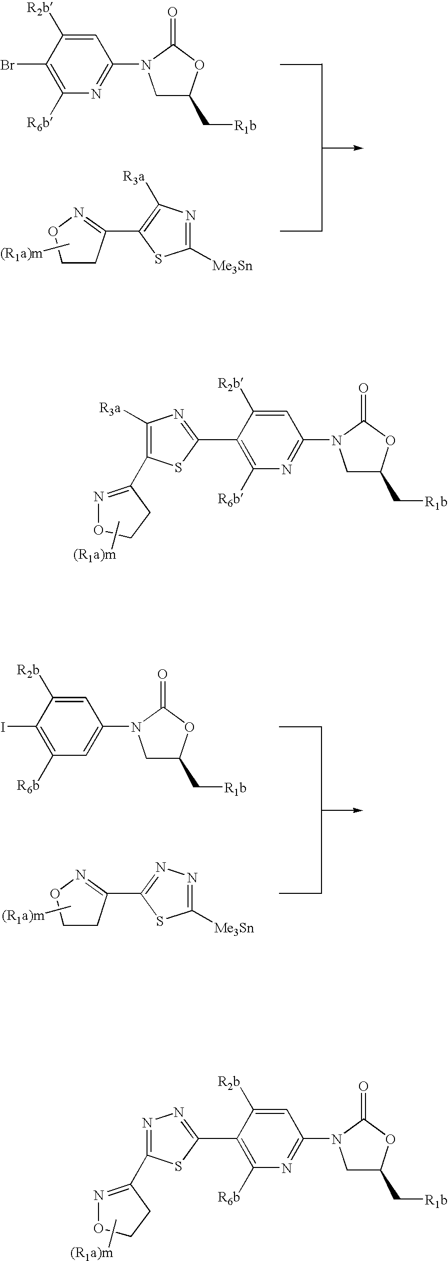 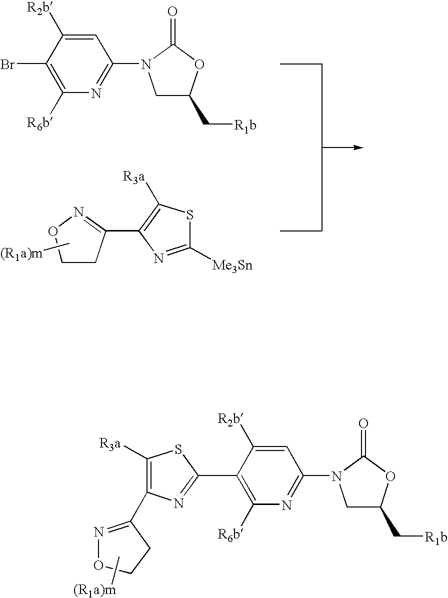
   the aryl isoxazolines and aryl oxazolidinones required as reagents for process b) or as intermediates for the preparation of reagents for process b) may be prepared by standard organic methods, for instance by methods analogous to those set out in process sections c) and h); methods for the introduction and interconversion of Groups X and X′ are well known in the art;
(c) by reaction of a (hetero)biary derivative (IIIa) or (IIIb) carbamate with an appropriately substituted oxirane (wherein 0, 1, or 2 of R₁a′-R₁a″″ are substitutents as defined for R₁a and the remainder are hydrogen) to form an oxazolidinone ring at the undeveloped aryl position; 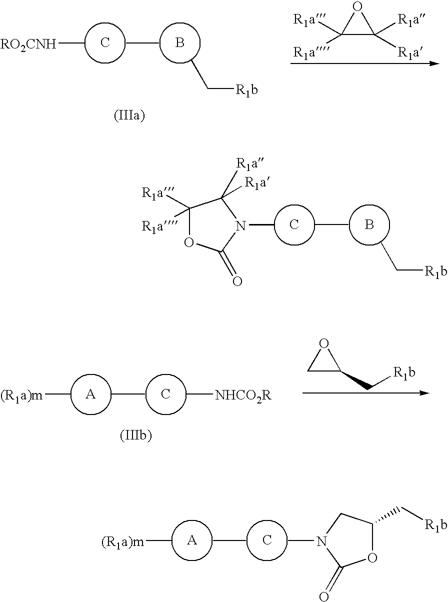
   variations on this process in which the carbamate is replaced by an isocyanate or by an amine or/and in which the oxirane is replaced by an equivalent reagent X—C(R₁a′)(R₁a″)C(R₁a′″)(O-optionally protected)(R₁a″″) or X—CH₂CH(O-optionally protected)CH₂R₁b where X is a displaceable group are also well known in the art; for example, 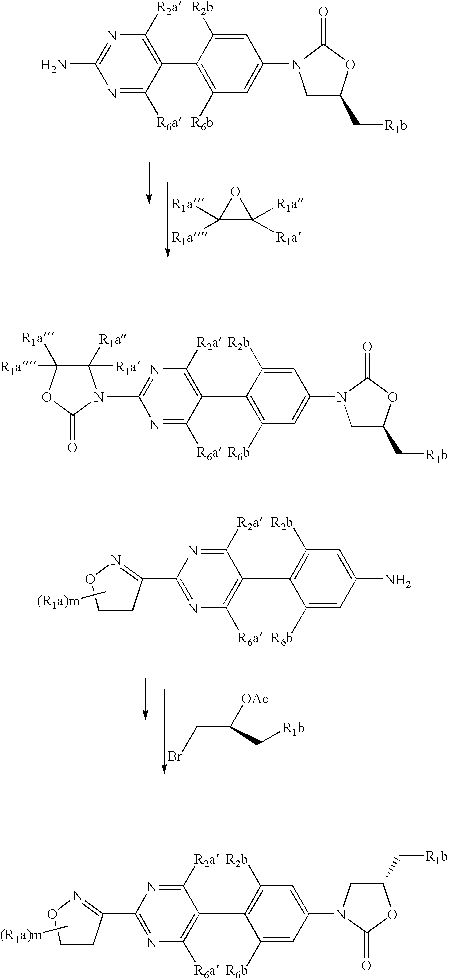
   (d) by reaction of a (hetero)biaryl derivative (IVa) or (IVb) to form an isoxazoline ring at the undeveloped aryl position; 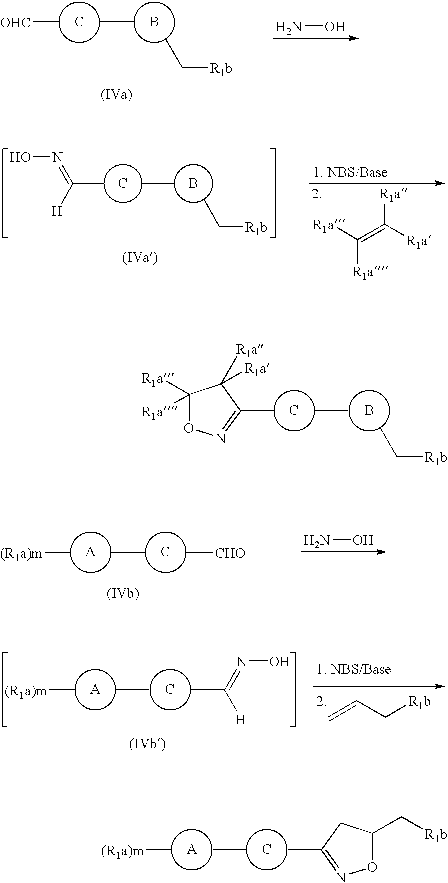
   variations on this process in which the reactive intermediate (a nitrile oxide IVa″ or IVb″) is
   obtained other than by oxidation of an oxime (IVa′) or (IVb′) are well known in the art; 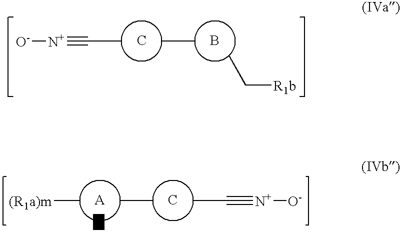
   for example, oxidation of an appropriately substituted biphenylcarboxaldehyde oxime in the presence of an appropriately substituted allyl derivative gives an isoxazoline of the required structure; 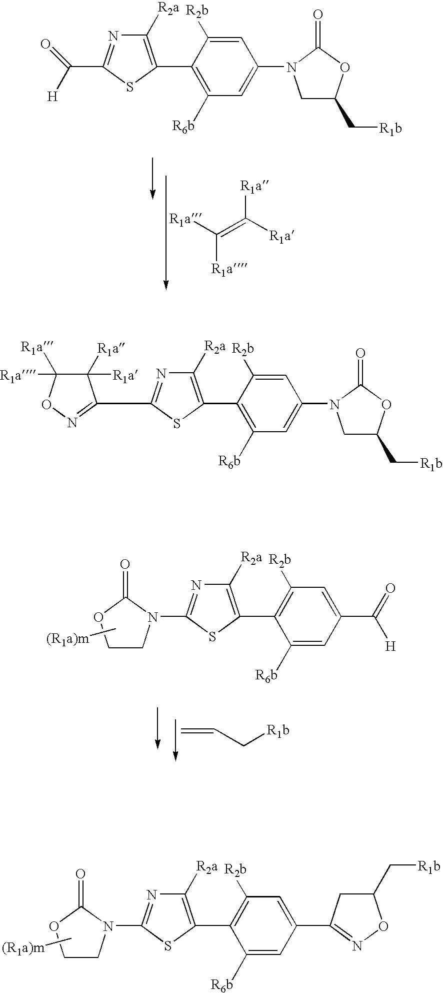
   enantioselective synthesis of 2-isoxazolines via asymmetric cycloaddition of nitrile oxides to olefins has been achieved by the use of chiral auxiliaries; for instance, when the alcohol is an allyl alcohol the desired stereochemistry at ring B can be obtained in reactions conducted in the presence of (R,R)-diisopropyl tartrate (or (S,S)-diisopropyl tartrate depending on the desired stereochemistry) as a chiral auxiliary (Yutaka Ukaji et al. Chem. Letters, 1993, 1847-1850); other chiral auxiliaries may also be employed with other olefins (see for instance Takahiko Akayama et al., Tet. Letters, 1992, 33, 5763-5766; and Jeffrey Stack et al., Tetrahedron, 1993, 49, 995-1008 and references therein); 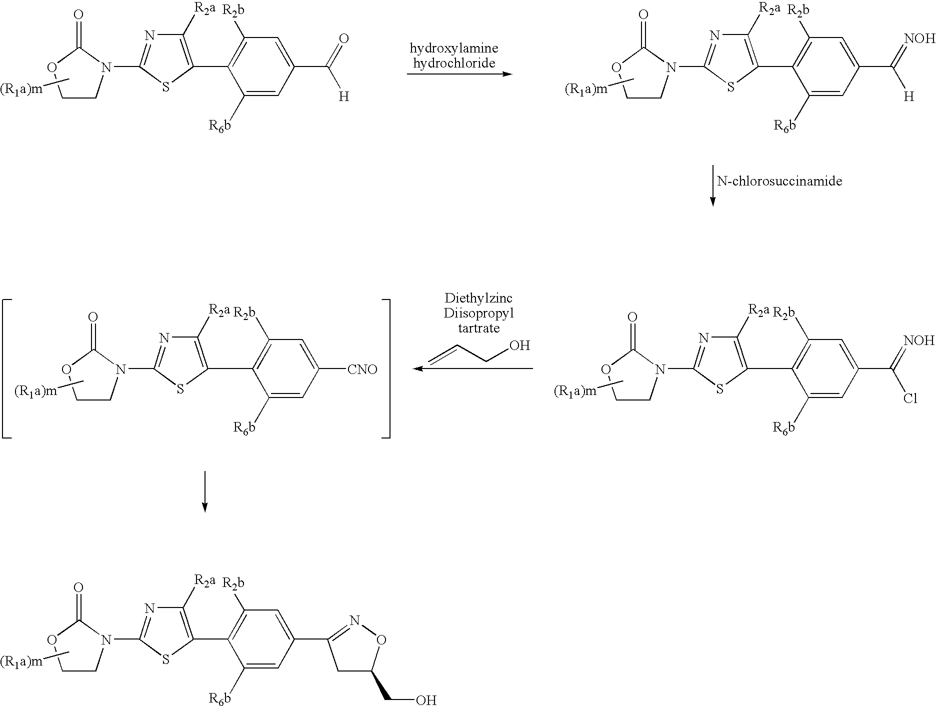
   (e) for HET2 as optionally substituted 1,2,3-triazoles, compounds of the formula (I) may be made by cycloaddition via the azide (wherein the substituent at the position of R1a in (I) is azide) to acetylenes, or to acetylene equivalents such as optionally substituted cyclohexa-1,4-dienes or optionally substituted ethylenes bearing eliminatable substituents such as arylsulfonyl;
(f) for HET2 as 4-substituted 1,2,3-triazole compounds of formula (I) may be made by reacting aminomethyloxazolidinones with 1,1-dihaloketone sulfonylhydrazones (Sakai, Kunihazu; Hida, Nobuko; Kondo, Kiyosi; Bull. Chem. Soc. Jpn., 59, 1986, 179-183; Sakai, Kunikazu; Tsunemoto, Daiei; Kobori, Takeo; Kondo, Kiyoshi; Hido, Noboko EP 103840 A2 19840328); for instance 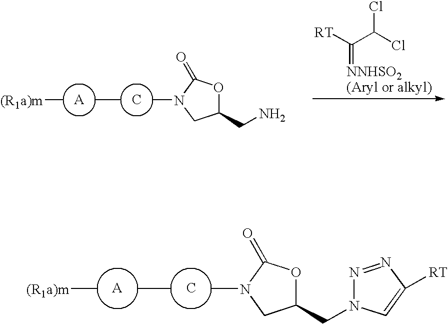
   (g) for HET2 as 4-substituted 1,2,3-triazole compounds of formula (I) may also be made by reacting azidomethyl oxazolidinones with terminal alkynes using Cu(I) catalysis in e.g. aqueous alcoholic solution at ambient temperatures to give 4-substituted 1,2,3-triazoles (V. V. Rostovtsev, L. G. Green, V. V. Fokin, and K. B. Sharpless, Angew. Chem. Int. Ed., 2002, 41, 2596-2599); for instance 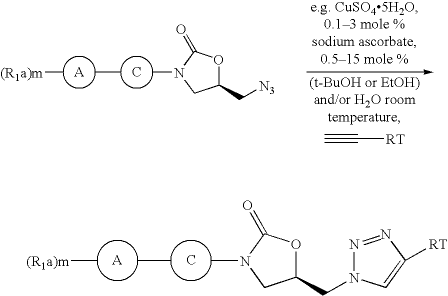
   (h) for HET2 as 4-halogenated 1,2,3-triazole compounds of formula (I) may also be made by reacting azidomethyl oxazolidinones with halovinylsulfonyl chlorides at a temperature between 0° C. and 100° C. either neat or in an inert diluent such as chlorobenzene, chloroform or dioxan; for instance as shown below.

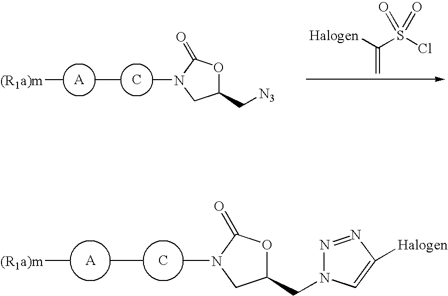

The removal of any protecting groups, the formation of a pharmaceutically-acceptable salt and/or the formation of an in vivo hydrolysable ester are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mg/kg⁻¹ to 20 mg/kg⁻¹ of a compound of this invention, the composition being administered 1 to 4 times per day. In another embodiment a daily dose of 5 mg/kg⁻¹ to 20 mg/kg⁻¹of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in vitro against standard Gram-positive organisms, which are used to screen for activity against pathogenic bacteria. Notably, the pharmaceutically-acceptable compounds of the present invention show activity against enterococci, pneumococci and methicillin resistant strains of S. aureus and coagulase negative staphylococci, together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system. The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well.

For example, the following results were obtained for the compound of Example 1:

| Organism | | MIC (μg/ml) |
|---|---|---|
| Staphylococcus aureus: | MSQS | 2 |
| | MRQR | 2 |
| Streptococcus pneumoniae | | 0.25 |
| Haemophilus influenzae | | 8 |
| Moraxella catarrhalis | | 2 |
| Enterococcus faecium | | 2 |
| Linezolid Resistant Streptococcus pneumoniae | | 4 |

MSQS = methicillin sensitive and quinolone sensitive
MRQR = methicillin resistant and quinolone resistant

Certain intermediates and/or Reference Examples described hereinafter are within the scope of the invention and may also possess useful activity, and are provided as a further feature of the invention.

The invention is now illustrated but not limited by the following Examples in which unless otherwise stated:—
(i) evaporations were carried out by rotary evaporation in vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Braker AM250 spectrometer operating at a field strength of 250 MHz; chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected]; optical rotations were determined at 589 nm at 20° C. for 0.1M solutions in methanol using a Perkin Elmer Polarimeter 341;
(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) in which the following abbreviations may be used

DMF is N,N-dimethylformamide; DMA is N,N-dimethylacetamide; TLC is thin layer chromatography; HPLC is high pressure liquid chromatography; MPLC is medium pressure liquid chromatography; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; ether is diethylether; EtOAc is ethyl acetate; MeOH is methanol; phosphoryl is (HO)₂—P(O)—O—; phosphiryl is (HO)₂—P—O—; Bleach is “Clorox” 6.15% sodium hypochlorite;
(viii) temperatures are quoted as ° C.

### EXAMPLE 1

### 3-(3-Fluoro-4-{2-(5RS)-[5-(hydroxymethyl)-4,5-dihydroisoxazol-3-yl]pyrimidin-5-yl}phenyl)-5-(5R)-(1H-1,2,3-triazol-1-ylmethyl)-1,3-oxazolidin-2-one

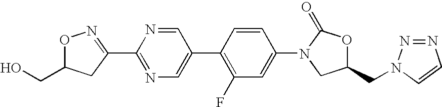

A mixture of (5R)-3-[3-fluoro-4-(trimethylstannyl)phenyl]-5-(1H-1,2,3-triazol-1-ylmethyl)-1,3-oxazolidin-2-one (906 mg, 2.13 mM), (5RS)-3-(5-bromopyrimidin-2-yl)-5-hydroxymethyl-4,5-dihydroisoxazole (500 mg, 1.94 mM), tris(dibenzylideneacetone) dipalladium (0)-chloroform adduct (200 mg, 0.194 mM, 0.1 equiv.), and tri-2-furylphosphine (89.9 mg, 0.388 mM, 0.2 equiv) was degassed and maintained under an atmosphere of nitrogen. The mixture was treated with anhydrous dioxane (10 ml) and heated at 95° C. for 24 hours. The reaction mixture was cooled and evaporated under reduced pressure. The involatile residue was purified by chromatography on silica gel [elution with 19:1 dichloromethane:methanol] to give the title compound (200 mg).

MS (APCI): 440 (M+1) for C₂₀H₁₈N₇O₄F

NMR (DMSO-d₆) δ: 3.26-3.56 (m, 4H); 3.96 (dd, 1H); 4.31 (t, 1H); 4.844.88 (m, 3H); 5.00 (brs, 1H); 5.19 (m, 1H); 7.44 (dd, 1H); 7.61 (dd, 1H); 7.76 (t, 1H); 7.77 (d, 1H); 8.19 (d, 1H); 9.11 (d, 2H).

The intermediates for this example were prepared as follows:

### Acetic acid (5R)-3-(3-fluorophenyl)-1,3-oxazolidin-2-on-5-ylmethyl ester

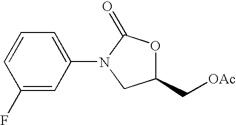

(5R)-3-(3-Fluorophenyl)-5-hydroxymethyl-1,3-oxazolidin-2-one (40 g, 0.189 M, see Upjohn WO 94-13649) was suspended by stirring in dry dichloromethane (400 mL) under nitrogen. Triethylamine (21 g, 0.208 M) and 4-dimethylaminopyridine (0.6 g, 4.9 mM) were added, followed by dropwise addition of acetic anhydride (20.3 g, 0.199 M) over 30 minutes, and stirring continued at ambient temperature for 18 hours. Saturated aqueous sodium bicarbonate (250 mL) was added, the organic phase separated, washed with 2% sodium dihydrogen phosphate, dried (magnesium sulfate), filtered and evaporated to give the desired product (49.6 g) as an oil.

MS (ESP): 254 (MH⁺) for C₁₂H₁₂FNO₄

NMR (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).
Acetic acid (5R)-3-(3-fluoro-4-iodo-phenyl)-1,3-oxazolidin-2-one-5-ylmethyl ester

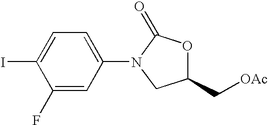

Acetic acid (5R)-3-(3-fluoro-phenyl)-1,3-oxazolidin-2-one-5-ylmethyl ester (15.2 g, 60 mM) was dissolved in a mixture of chloroform (100 mL) and acetonitrile (100 mL) under nitrogen, and silver trifluoroacetate (16.96 g, 77 mM) added. Iodine (18.07 g, 71 mM) was added in portions over 30 minutes to the vigorously stirred solution, and stirring continued at ambient temperature for 18 hours. As reaction was not complete, a further portion of silver trifluoroacetate (2.64 g, 12 mM) was added and stirring continued for 18 hours. After filtration, the mixture was added to sodium thiosulfate solution (3%, 200 mL) and dichloromethane (200 mL), and the organic phase separated, washed with sodium thiosulfate (200 mL), saturated aqueous sodium bicarbonate (200 mL), brine (200 mL), dried (magnesium sulfate), filtered and evaporated. The crude product was suspended in isohexane (100 mL), and sufficient diethyl ether added to dissolve out the brown impurity while stirring for 1 hour. The product was isolated by filtration to give the title compound (24.3 g) as a cream solid.

MS (ESP): 380 (MH⁺) for C₁₂H₁₁FINO₄

NMR (DMSO-d₆) δ: 2.03 (s, 3H); 3.82 (dd, 1H); 4.15 (t, 1H); 4.24 (dd, 1H); 4.30 (dd, 1H); 4.94 (m, 1H); 7.19 (dd, 1H); 7.55 (dd, 1H); 7.84 (t, 1H).
5R)-3-(3-Fluoro-4-iodophenyl)-5-hydroxymethyl-1,3-oxazolidin-2-one

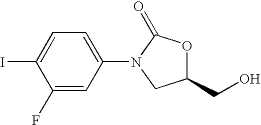

A solution of acetic acid (5R)-3-(3-fluoro-4iodophenyl)-1,3-oxazolidin-2-one-5-ylmethyl ester (30 g, 79 mM) in a mixture of methanol (800 mL) and dichloromethane (240 mL) was treated at ambient temperature with potassium carbonate (16.4 g, 0.119 mM) for 25 minutes, then immediately neutralised by the addition of acetic acid (10 mL) and water (500 mL). The precipitated product was filtered, washed with water, and then dissolved in dichloromethane (1.2 L) to give a the solution that was washed with saturated sodium bicarbonate and then dried (magnesium sulfate). The solution of product was filtered and evaporated to dryness to give the title compound (23 g).

MS (ESP): 338 (MH⁺) for C₁₀H₉FINO₃

NMR (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1M); 7.81 (t, 1H). (5R)-5-Azidomethyl-3-(3-fluoro-4-iodophenyl)-1,3-oxazolidin-2-one 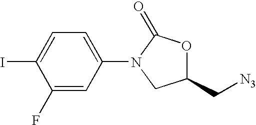
Note: This intermediate compound is considered likely to be explosive so should be used without isolation or treated with extreme caution, particularly at high temperatures.

A stirred solution of (5R)-3-(3-fluoro-4-iodophenyl)-5-hydroxymethyl-1,3-oxazolidin-2-one (55.8 g) and triethylamine (46.1 mL) in dry dichloromethane (800 mL) under an atmosphere of dry nitrogen was maintained below room temperature by an ice-bath and treated dropwise with methanesulfonyl chloride (17.9 mL). The stirred reaction mixture was allowed to warm to room temperature during 3 hours and then washed sequentially with water and brine and then dried (Na₂SO₄). Solvent was removed under reduced pressure to give the intermediate mesylate as a yellow solid (68 g) that was used without further purification.

A stirred solution in DMF (800 mL) of a mixture of the intermediate mesylate (68 g) and sodium azide (32.3 g) was heated at 75° C. overnight. The mixture was allowed to cool to room temperature, diluted with water, and extracted twice with ethyl acetate. The combined extracts were washed sequentially with water and brine, and then dried (Na₂SO₄). Solvent was removed under reduced pressure to give a yellow oil that was purified by column chromatography on silica-gel [elution with ethyl acetate:hexanes (1:1)] to give the product azide as an off-white solid (49 g). The product could be further purified by trituration with ethyl acetate/hexanes.

¹H-NMR (DMSO-d₆) δ: 3.57-3.64 (dd, 11l); 3.70-3.77 (dd, 1H); 3.81-3.87 (dd, 1H); 4.06 (t, 1H); 4.78-4.84 (m, 1H); 7.05-7.09 (ddd, 1H); 7.45 (dd, 1H); 7.68-7.74 (dd, 1H).
(5R)-3-(3-Fluoro-4iodophenyl)-5-(1H-1,2,3-triazol-1-ylmethyl)-1,3-oxazolidin-2-one

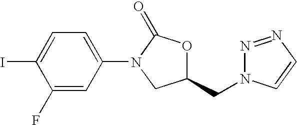

A stirred solution in dioxan (300 mL) of a mixture of the (5R)-5-azidomethyl-3-(3-fluoro-4-iodophenyl)-1,3-oxazolidin-2-one (30 g) and bicyclo[2.2.1]heptadiene (30 mL) was heated under reflux overnight. The mixture was allowed to cool to room temperature and then evaporated to dryness under reduced pressure to give a brown solid. The brown solid was purified by column chromatography on silica-gel [elution with a gradient from 98:2 to 95:5 methanol:chloroform] to give the product triazole as a pale yellow solid (20 g). The product could be further purified by trituration with dichloromethane/hexanes (1:1) to give an off-white solid.

¹H-NMR (DMSO-d₆) δ: 3.86-3.92 (dd, 1H); 4.23 (t, 1H); 4.83 (d, 2H); 5.11-5.19 (m, 1H); 7.12-7.16 (dd, 1H); 7.47-7.51 (dd, 1H); 7.76 (s, 1H); 7.79-7.85 (dd, 1H); 8.16 (s, 1H).
(5R)-3-[3-Fluoro-4-(trimethylstannyl)phenyl]-5-(1H-1,2,3-triazol-1-ylmethyl)-1,3-oxazolidin-2-one

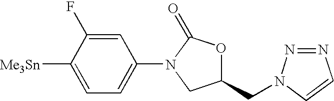

A mixture of (5R)-3-(3-fluoro-4-iodophenyl)-5-(1H-1,2,3-triazol-1-ylmethyl)-1,3-oxazolidin-2-one (5.39 g, 13.9 mmol) and hexamethylditin (5 g, 15.3 mmol) in dioxane (50 ml) under an atmosphere of nitrogen was treated with dichlorobis(triphenylphoshine)palladium (II) (487 mg, 0.69 mmol) and then stirred at 90° C. under an atmosphere of nitrogen for 90 minutes. Silica gel (5 g) was added then the solvent removed under reduced pressure. The residual powder was placed on top of a silica gel column (100 g) and eluted (1% methanol in dichloromethane to 2.5% methanol in dichloromethane gradient) to give the desired product (4.545 g).

MS (ESPI 423, 425, 427 (MH+) for C₁₅H₁₉FN₄O₂Sn.

¹H-NMR (DMSO-d₆) δ: 0.32 (s, 9H); 3.90 (dd, 1H); 4.25 (t, 1H); 4.85 (d, 2H); 5.16 (m, 1H); 7.26 (dd, 1H); 7.33 (dd, 1H); 7.41 (dd, 1H); 7.78 (s, 1H); 8.18 (s, 1H).
(5RS)-3-(5-bromopyrimidin-2-yl)-5-hydroxymethyl-4,5-dihydroisoxazole

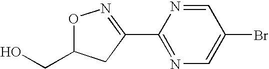

A solution of methylmagnesium chloride in tetrahydrofuran (3M; 13.7 mL, 41.04 mmol) was added slowly to a stirred solution of 2-iodo-5-bromopyrimidine (7.7 g, 27.4 mmol) in tetrahydrofuran (70 mL) at −78° C. The resultant yellow solution was stirred at −78° C. for 30 min and then treated with N,N-dimethylformamide (21.2 ml, 273 mmol). The solution was allowed to warmed slowly and then stirred at room temperature for 2 hours to give a crude solution of the intermediate 5-bromopyrimidine 2-carboxaldehyde that was used without further purification. The crude solution of intermediate 5-bromopyrimidine 2-carboxaldehyde was diluted with methanol (50 ml) and water (50 ml), treated with hydroxylamine hydrochloride (3.77 g, 54.7 mmol) and sodium carbonate (1.74 g, 16.4 mmol), and then stirred at room temperature for 1 hour. The reaction mixture was partitioned between dichloromethane (200 ml) and water (150 ml) and the organic phase was separated. The aqueous phase was washed with dichloromethane (2×200 ml) and the organic phases were combined, dried over sodium sulfate, filtered, and concentrated in vacuo to give an involatile residue of crude intermediate 5-bromopyrimidine 2-carboxaldehyde oxime that was used without further purification. A stirred mixture of the involatile residue of the unpurified 5-bromopyrimidine 2-carboxaldehyde oxime and allyl alcohol (7.5 ml, 109 mmol) in tetrahydrofuran (100 ml) was treated with bleach (Clorox, 6.15% NaOCl; 195 ml, 136 mmol) and then stirred at room temperature for two hours. The reaction mixture was followed by extraction with tetrahydrofuran (2×250 ml) and the organic phases were combined and concentrated. The involatile residue was purified by chromatography on silica gel [elution with 1:1 hexane:ethyl acetate) to give the title compound (1.5 g).

MS (APCI): 258 (M+1) for C₈H₈N₃O₂Br

NMR (DMSO-d₆) δ: 3.20-3.56 (m, 4H); 4.82 (m, 1H); 5.02 (m, 1H); 9.09 (s, 2H).

### EXAMPLE 2

### 3-(3-Fluoro-4-{2-(5RS)-[5-(hydroxymethyl)-4,5-dihydroisoxazol-3-yl]pyrimidin-5-yl}phenyl)-5-(5S)-(acetamidomethyl)-1,3-oxazolidin-2-one

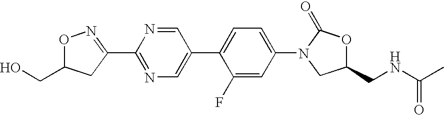

A mixture of (5S) 3-[3-fluoro-4-(trimethylstannyl)phenyl]-5-(acetamidomethyl)-1,3-oxazolidin-2-one (177 mg, 0.426 mM) (Dong A Pharm. Co. Ltd., WO 01/94342), (5RS)-3-(5-bromopyrimidin-2-yl)-5-hydroxymethyl-4,5-dihydroisoxazole (100 mg, 0.387 mM), tris(dibenzylidineacetone) dipalladium (0)-chloroform adduct (40 mg, 0.039 mM, 0.1 equiv.), and tri-2-furylphosphine (18 mg, 0.077 mM, 0.2 equiv) ) was degassed and maintained under an atmosphere of nitrogen. The mixture was treated with anhydrous dioxane (5 ml) and heated at 95° C. for 12 hours. The reaction mixture was cooled and evaporated under reduced pressure. The involatile residue was purified by chromatography on silica gel [elution with 19:1 dichloromethane:methanol] to give the title compound (30 mg).

MS (APCI): 430 (M+1) for C₂₀H₂₀N₅O₅F

NMR (DMSO-d₆) δ: 1.87 (s, 3H); 3.44-3.80 (m, 7H); 3.81 (dd, 1H); 4.16 (t, 1H); 4.80-4.85 (m, 2H); 5.05 (t, 1H); 7.49 (dd, 1H); 7.68 (dd, 1H); 7.81 (t, 1H); 8.26 (t, 1H); 9.11 (d, 2H).

### EXAMPLE 3

### (5R)-3-(3-Fluoro-4-{2-[(5R)-5-(hydroxymethyl)-2-oxo-1,3-oxazolidin-3-yl]-1,3-thiazol-5-yl}phenyl)-5-(1H-1,2,3-triazol-1-ylmethyl)-1,3-oxazolidin-2-one

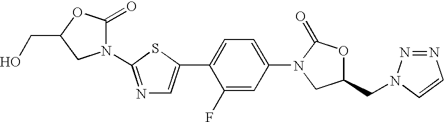

(5R)-3-[(3-Fluoro-4-(trimethylstannyl)phenyl)]-5-[(1H-1,2,3-triazol-1-ylmethyl)-1,31]-oxazolidin-2-one (673.0 mg, 1.58 mM), (5R)-3-(5-bromo-1,3-thiazol-2-yl)-5-(hydroxymethyl)-1,3-oxazolidin-2-one (400 mg, 1.43 mM), tris(dibenzylidineacetone) dipalladium (0)-chloroform adduct (149.0 mg, 0.143 mM, 0.1 equiv.), tri-2-furylphosphine (66.8 mg, 0.288 mM, 0.2 equiv) were placed in a flask. The solids were degassed and placed under nitrogen. Anhydrous dioxane (10 ml) was added and the suspension was heated at 95° C. for 24 hours. The reaction mixture was cooled and the solvent was evaporated. The residue was chromatographed on silica gel eluting with 5% methanol in dichloromethane to give title compound (20 mg).

MS (APCI): 461 (M+1) for C₁₉H₁₇N₆O₅SF

¹H NMR (DMSO-d₆) δ: 3.58-3.66 (m, 1H); 3.70-3.77 (m, 1H); 3.93 (dd, 1H); 4.05 (dd, 1H); 15 4.28 (m, 2H); 4.86 (m, 3H); 5.16 (m, 1H); 5.28 (t, 1I); 7.34 (dd, 1H); 7.54 (dd, 1H); 7.76 (s, 1H); 7.79 (s, 1H); 7.90 (s, 1H), 8.14 (s, 1H).

Intermediates for the above were prepared as follows:
(5R)-3-(5-Bromo-1,3-thiazol-2-yl)-5-(hydroxymethyl)-1,3-oxazolidin-2-one 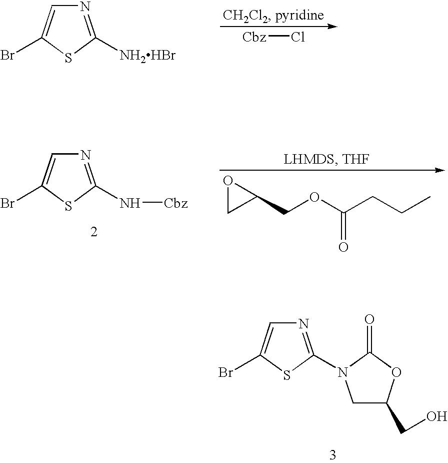
benzyl chloroformate (18 g, 0.106 mol) was added dropwise to a solution of 2-amino-5-bromothiazole monohydrobromide (25 g, 0.096 mol) in dichloromethane (500 mL) and pyridine (22.8 g, 0.288 mol) cooled in an ice bath, followed by warming to ambient temperature over 16 hours. Reaction mixture was concentrated to remove most of the solvent, diluted with water and stirred for 30 min. Filtration of the mixture provided the product 2 as a cream solid (26 g, 86.7%). Lithium bis(trimethylsilyl)amide (1.0 M in THF, 52 mL, 0.052 mol) was added dropwise to a solution of (5-bromo-thiazol-2-yl)-carbamic acid benzyl ester 2 (15.5 g, 0.0495 mol) in anhydrous TBF (500 mL) cooled to −78° C. (suspension) under a nitrogen atmosphere, followed by warming to 0° C. for 15 minutes. The reaction mixture was cooled to −78° C. and (R)-(−)-glycidyl butyrate (7.4 mL, 0.052 mol) was added dropwise, warmed slowly to ambient temperature over night. The reaction mixture was quenched with water (250 mL) and diluted with EtOAc (1 L). The organic layer was separated and washed with water, brine, dried over sodium sulfate and concentrated. The crude product was triturated with dichloromethane/hexanes to yield 3 (4 g, 29%) as a pale yellow solid. The filtrate was concentrated and dissolved in MeOH. Sodium methoxide (0.2 g) was added and the mixture was stirred for 15 min, then quenched with water, concentrated to remove MeOH, and extracted with dichloromethane. The organic layer was separated and washed with water, brine, dried over sodium sulfate and concentrated. The residue was triturated with dichloromethane/hexanes to yield 3 (1.5 g, 11%) as a pale yellow solid. Yield=4 g+1.5 g (40%).
(5R)-3-[(3-Fluoro-4-(trimethylstannyl)phenyl)]-5-[(1H-1,2,3-triazol-1-ylmethyl)-1,3]-oxazolidin-2-one

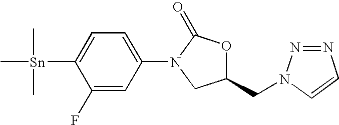

A mixture of (5R)-3-(3-fluoro-4-iodophenyl)-5-[(1H-1,2,3-triazol-1-yl)methyl]-1,3-oxazolidin-2-one (4 g, 10.31 mM) (see Example 1) and bis(triphenylphospine)palladium(II) chloride (0.72 g, 0.10 mM) was degassed and maintained under argon. The reaction mixture was treated with dioxane (60 mL) and then with hexamethylditin (5.00 g, 15.5 mM) and the reaction was degassed again and maintained under argon. The reaction mixture was heated at 100° for 3 hour. The cool reaction mixture was cooled and the solvent was evaporated. The residue was chromatographed on silica gel eluting with 5% methanol in dichloromethane to give the title compound (2.8 g).

MS (ESP): 426 (MH⁺) for C₁₅H₁₉FN₄O₂Sn

NMR (DMSO-d₆) δ: 0.3 (t, 9H); 3.88 (dd, 1H); 4.23 (t, 1H); 4.83 (d, 2H); 4.15 (m, 1H); 7.24 (dd, 1H); 7.33 (dd, 1H); 7.44 (dd, 1H); 7.76 (d, 1H); 8.17 (d, 1H).

### EXAMPLE 4

### N-{[3-(3-Fluoro-4-{6-[5-(hydroxymethyl)-4,5-dihydro-3-isoxazolyl]-3-pyridazinyl}phenyl)-2-oxo-1,3-oxazolidin-5-yl]methyl}acetamide

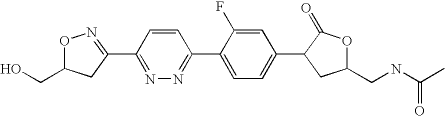

(3-(6-Chloro-3-pyridazinyl)-4,5-dihydro-5-isoxazolyl]methanol (see Example 5 below, 32 mg, 0.15 mmol), N-({3-[3-fluoro-4-(4,4,5,5-tetramethyl-1,3,2-dioxaborolan-2-yl)phenyl]-2-oxo-1,3-oxazolidin-5-yl}methyl)acetamide (prepared from N-{[(5S)-3-(3-Fluoro-4-iodophenyl)-2-oxo-1,3-oxazolidin-5-yl]methyl}acetamide by an analogous method to that used in Example 5; 60 mg, 0.17 mmol), potassium carbonate (68 mg, 0.51 mmol), and tetrakis(triphenylphosphino)palladium(0) (23 mg, 0.02 mmol) were combined and suspended in DMF (3 ml) and water (0.5 ml). The mixture was heated at 80 ° C. for 2 hours, then diluted with water to 20 ml. The solids were collected, rinsed with water and resuspended in warm DMSO (1 ml). The suspension was diluted with dichloromethane (3 ml) and ether (1 ml). The solid was collected, rinsed with ether and methanol, and dried in vacuo to give the pure product as a light yellow solid, 10 mg.

MS (APCI): 430 (M+1) for C₂₀H₂₀N₅O₅F.

¹H-NMR (DMSO-d₆) δ: 1.84 (s, 3H); 3.36-3.58 (m, 6H); 3.82 (dd, 1H); 4.25 (t, 1H); 4.78 (m, 1H); 4.86 (m, 1H); 5.08 (t, 1H); 7.55 (d, 1H); 7.75 (d, 1H); 8.11 (dd, 1H); 8.21 (dd, 1H); 8.25 (t, 1H).

¹⁹F—NMR (DMSO-d₆) δ: −114.8 ppm.

The intermediates for this compound were prepared as follows:
Acetic acid (SR)-3-(3-fluoro-phenyl)-2-oxo-oxazolidin-5-ylmethyl ester

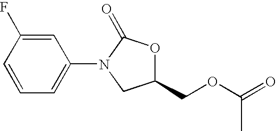

(5R)-3-(3-Fluorophenyl)-5-hydroxymethyloxazolidin-2-one (40 g, 0.189 mol, see Upjohn WO 94-13649) was suspended by stirring in dry dichloromethane (400 ml) under nitrogen. Triethylamine (21 g, 0.208 mol) and 4dimethylaminopyridine (0.6 g, 4.9 mmol) were added, followed by dropwise addition of acetic anhydride (20.3 g, 0.199 mol) over 30 minutes, and stirring continued at ambient temperature for 18 hours. Saturated aqueous sodium bicarbonate (250 ml) was added, the organic phase separated, washed with 2% sodium dihydrogen phosphate, dried (magnesium sulfate), filtered and evaporated to give the desired product (49.6 g) as an oil.

MS (ESP): 254 (MH⁺) for C₁₂H₁₂FNO₄

NMR (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).
Acetic acid (5R)-3-(3-fluoro-4-iodo-phenyl)-2-oxo-oxazolidin-5-ylmethyl ester

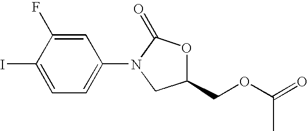

Acetic acid (5R)-3-(3-fluoro-phenyl)-2-oxo-oxazolidin-5-ylmethyl ester (15.2 g, 60 mmol) was dissolved in a mixture of chloroform (100 ml) and acetonitrile (100 ml) under nitrogen, and silver trifluoroacetate (16.96 g, 77 mmol) added. Iodine (18.07 g, 71 mmol) was added in portions over 30 minutes to the vigorously stirred solution, and stirring continued at ambient temperature for 18 hours. As reaction was not complete, a further portion of silver trifluoroacetate (2.64 g, 12 mmol) was added and stirring continued for 18 hours. After filtration, the mixture was added to sodium thiosulfate solution (3%, 200 ml) and dichloromethane (200 ml), and the organic phase separated, washed with sodium thiosulfate (200 ml), saturated aqueous sodium bicarbonate (200 ml), brine (200 ml), dried (magnesium sulfate), filtered and evaporated. The crude product was suspended in isohexane (100 ml), and sufficient diethyl ether added to dissolve out the brown impurity while stirring for 1 hour. Filtration gave the desired product (24.3 g) as a cream solid.

MS (ESP): 380 (MH⁺) for C₁₂H₁₁FINO₄

NMR (DMSO-d₆) δ: 2.03 (s, 3H); 3.82 (dd, 1H); 4.15 (t, 1H); 4.24 (dd, 1H); 4.30 (dd, 1H); 4.94 (m, 1H); 7.19 (dd, 1H); 7.55 (dd, 1H); 7.84 (t, 1H).
(5R)-3-(3-Fluoro4-iodophenyl)-5-hydroxymethyloxazolidin-2-one

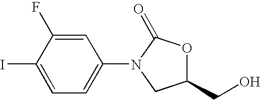

Acetic acid (5R)-3-(3-fluoroiodophenyl)-2-oxo-oxazolidin-5-ylmethyl ester (30 g, 79 mmol) was treated with potassium carbonate (16.4 g, 0.119 mmol) in a mixture of methanol (800 ml) and dichloromethane (240 ml) at ambient temperature for 25 minutes, then immediately neutralised by the addition of acetic acid (10 ml) and water (500 ml). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).
[(5R)-3-(3-Fluoro-4-iodophenyl)-2-oxo-1,3-oxazolidin-5-yl]methyl methanesulfonate

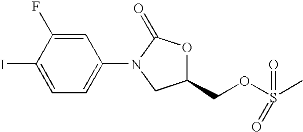

(5R)-3-(3-Fluoro-4-iodophenyl)-5-(hydroxymethyl)-1,3-oxazolidin-2-one (25.0 g, 74.2 mmol) was stirred in methylene chloride (250 ml) at 0° C. Triethylamine (10.5 g, 104 mmol) was added followed by methanesulfonyl chloride (11.2 g, 89.0 mmol) and the reaction was stirred overnight, slowly warming to room temperature. The yellow solution was diluted with sodium bicarbonate and the compound was extracted using methylene chloride (3×250 ml). The organic layer was dried (magnesium sulfate), filtered and concentrated to give the desired product as a light yellow solid (30.3 g).

MS (ESP): 416 (M⁺) for C₁₁H₁₁FINO₅S

¹H-NMR (DMSO-d₆): 3.24 (s, 3H); 3.82 (dd, 1H); 4.17 (t, 1H); 4.43-4.52 (m, 2H); 4.99-5.03 (m, 1H); 7.21 (dd, 1H); 7.55 (dd, 1H); 7.83 (t, 1H).
(5R)-5-(Azidomethyl)-3-(3-fluoro-4-iodophenyl)-1,3-oxazolidin-2-one

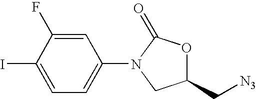

[(5R)-3-(3-Fluoro-4-iodophenyl)-2-oxo-1,3-oxazolidin-5-yl]methyl methanesulfonate (6.14 g, 14.7 mmol) was dissolved in N,N-dimethylformamide (50 ml). Sodium azide (1.92 g, 29.6 mmol) was added and the reaction was stirred at 75° C. overnight. The yellow mixture was poured into half-saturated sodium bicarbonate and extracted using ethyl acetate. The organic layer was washed three times with water, dried (magnesium sulfate), filtered, and concentrated to give the title compound as a yellow solid (4.72 g).

MS (ESP): 363 (MH⁺) for C₁₀H₈FIN₄O₂

¹H-NMR (DMSO-d₆): 3.72-3.82 (m, 3H); 4.14 (t, 1H); 4.89-4.94 (m, 1H); 7.22 (dd, 1H); 7.57 (dd, 1H); 7.83 (t, 1H).
N-{[(5S)-3-(3-Fluoro-4-iodophenyl)-2-oxo-1,3-oxazolidin-5-yl]methyl}acetamide

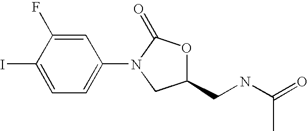

(5R)-5-(Azidomethyl)-3-(3-fluoro-4-iodophenyl)-1,3-oxazolidin-2-one (5.00 g, 0.014 mol) was suspended in thioacetic acid (10 ml) and the solution was stirred under nitrogen at room temperature for approximately 16 h. The resulting suspension was concentrated under vacuum. The crude product was crystallized from methanol/acetone, then further purified by chromatography on silica gel using dichloromethane to give 3.71 g of the title product as a white solid.

MS (ESP): 379 (MH⁺) for C₁₂H₁₂FIN₂O₃

¹H-NMR(500 MHz) (DMSO-d₆): 1.86 (s, 3H); 3.45 (t, 2H); 3.76 (dd, 1H); 4.14 (t, 1H); 4.78 (m, 1H); 7.22 (dd, 1H); 7.58 (dd, 1H); 7.87 (t, 1H); 8.28 (t, 1H).

### EXAMPLE 5

### 3-(3-Fluoro-4-{6-[5-(hydroxymethyl)-4,5-dihydro-3-isoxazolyl]-3-pyridazinyl}phenyl)-5-(1H-1,2,3-triazol-1-ylmethyl)-1,3-oxazolidin-2-one

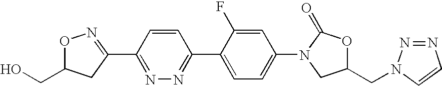

[3-(6-Chloro-3-pyridazinyl)-4,5-dihydro-5-isoxazolyl]methanol (430 mg, 2.02 mmol), 3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one, (400 mg, 1.10 mmol), potassium carbonate (415 mg, 3.0 mmol), and tetrakis(triphenylphosphino)palladium(0) (230 mg, 0.2 mmol) were combined and suspended in DMF (5 ml) and water (0.5 ml). The mixture was heated at 80° C. for 2 hours then diluted with water to 20 ml. The solids were collected, rinsed with water and re-suspended in warm DMSO (2 ml). The suspension was diluted with dichloromethane (3 ml) and ether (1 ml). The solid was collected, rinsed with ether and methanol, and dried in vacuo to give a 90% pure product as a light yellow solid. The product was repurified by preparative HPLC using a gradient from 5 to 95% acetonitrile/water containing 0.1% trifluoro acetic acid over 14 minutes. Fractions that contained the desired product were combined, concentrated and lyophilized to give 10 mg of the title compound.

MS (APCI): 440 (M+1) for C₂₀H₁₈N₇₄F.

¹H-NMR (DMSO-d₆) δ: 3.56-3.66 (m, 4H); 3.92 (m, 1H); 4.31 (t, 1H); 4.86 (m, 3H); 5.19 (m, 1H); 7.50 (d, 1); 7.63 (d, 1H); 7.77 (s, 1H); 8.10 (dd, 1H); 8.20 (dd, 1H); 8.25 (t, 1H).

¹⁹F-NMR (DMSO-D₆) δ: −114.7 ppm; −73.7 ppm (trifluoroacetate).

The intermediates for the above were prepared as follows:
(5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one 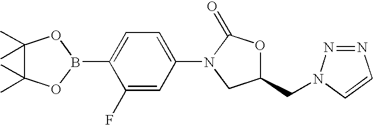
(5R)-3-(3-Fluoro-4-iodophenyl)-5-(1H-1,2,3-triazol-1-ylmethyl)-1,3-oxazolidin-2-one (see Example 1, 2 g, 5.15 mmol), bis(pinacolato)diboron, 2.62 g (10.3 mmol), potassium acetate, 2.5 g (25.5 mmol), and 1,1′-[bis(diphenylphosphino)ferrocene]dichloropalladium(II) dichoromethane complex, 0.38 g (0.52 mmol) were suspended in DMSO (15 ml). The mixture was heated at 80° C. for 40 minutes to give a clear black solution. Ethyl acetate (150 ml) was then added and the mixture was filtered through celite, washed with saturated brine (2×100 ml), dried over sodium sulfate and evaporated. The residue was purified by chromatography (silica gel, 40 to 100% ethyl acetate in hexane, followed by 1-5% acetonitrile in ethyl acetate) to give the product as a crystalline tan solid, 1.97g (98%).

¹H-NMR (300 MHz. DMSO-d₆) δ: 1.28 (s, 12H), 3.91 (dd, 1H); 4.23 (t, 1H); 4.83 (d, 2H); 5.14 (m, 1H); 7.27 (dd, 1H); 7.37 (dd, 1H); 7.62 (t, 1H); 7.75 (s, 1H); 8.16 (s, 1H).
3-(Chloro-3-pyridazinyl)-4.5-dihydro-5-isoxazolyl]methanol

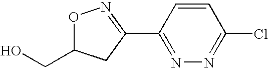

To a solution of 6-chloro-3-pyridazinecarbaldehyde oxime (1.9 g; 12.1 mmol) and allyl alcohol (2.5 mL, 36 mmol) in 10 mL THF there was dropped 72 mL of an aqueous sodium hydrochlorite solution (37.5%, chlorox) over 15 minutes during which a precipitate formed. The reaction was stirred for another 2 hours and mixed with 200 ml of a methanol/dichloromethane mixture 5/95 (v/v). The organic layer was separated and the aqueous layer was washed again with the organic solvent system (2×100 mL). The organic layers were combined, dried over anhydrous sodium sulfate and concentrated. The crude product was purified by chromatography using an isocratic eluent of ethylacetate/hexanes (1:1) to give the title compound.

MS (APCI): 214 (M+1) for C₈H₈N₃O₂Cl.

¹H-NMR (DMSO-d₆) δ: 3.06-3.66 (m, 4H); 4.71 (m, 1H); 7.50 (d, 1H); 7.63 (d, 1H).
6-Chloro-3-pyridazinecarbaldehyde oxime

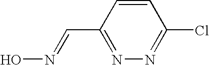

6-Chloro-3-pyridazinecarbaldehyde (1.9 g, 13.2 mmol) was suspended in 20 mL of aqueous methanol (1:1) and hydroxylamine hydrochloride (1.2 g, 17.1 mmol) and sodium carbonate (1.1 g, 10.0 mmol) was added. A precipitate formed immediately and the reaction reached completion after 20 minutes. The mixture was washed with ethyl acetate (20 mL), separated and the aqueous layer extracted with ethyl acetate (2×10 mL). The organic layer were combined, dried over sodium sulfate, filtered and concentrated to dryness to give the pure title compound (95% yield).

¹H-NMR (DMSO-d₆) δ: 7.78 (d, 1H); 7.94 (d, 1H); 8.61 (s, 1H); 12.60 (s, 1H).
6-chloro-3-pyridazinecarbaldehyde

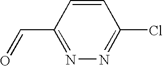

A solution of ethyl 6-chloro-3-pyridazinecarboxylate (3.3 g, 17.6 mmol) in 150 mL of anhydrous tetrahydrofuran was cooled in an ice bath. Diisobutyl aluminium hydride (35 mL, 1 M in toluene, 35 mmol) was slowly added over 5 minutes. After 10 minutes, thin layer chromatography (40% ethylacetate in hexanes as eluent) showed consumption of all starting material and the reaction was quenched with 20 mL of ice water, neutralized with 1N aqueous HCl and saturated sodium bicarbonate solution. The solution was extracted with dichloromethane (3×150 mL). The organic layers were combined, dried over anhydrous sodium sulfate and concentrated to dryness to give 2.2 g (85%) of the title compound.

¹H-NMR (DMSO-d₆) δ: 8.10 (s, 2H); 10.10 (s, 1H).

### EXAMPLE 6

### (5R)-3-(3-Fluoro-4-{2-[5-(hydroxymethyl)-4,5-dihydroisoxazol-3-yl]pyrimidin-5-yl}phenyl)-5-{[4-(fluoromethyl)-1H-1,2,3-triazol-1-yl]methyl}-1,3-oxazolidin-2-one

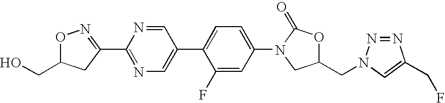

[3-(5-Bromopyrimidin-2-yl)4,5-dihydro-isoxazol-5-yl]-methanol (204 mg, 0.790 mmol), (5R)-3-[3-fluoro-4-(4,4,5,5-tetramethyl-1,3,2-dioxaborolan-2-yl)phenyl]-5-{[4-(fluoromethyl)-1H-1,2,3-triazol-1-yl]methyl}-1,3-oxazolidin-2-one (0.332 mg, 5.15 mmol), potassium carbonate (327 mg, 2.37 mmol), and tetrakis(triphenylphosphino) palladium(0) (91 mg, 0.079 mmol) were combined and suspended in DNT (4 ml) and water (0.4 ml). The mixture was heated at 80° C. for 2 hours, then diluted with water to 7 ml. The solids were collected, rinsed with water and resuspended in warm DMSO (3 ml). The suspension was diluted with dichloromethane (5 ml) and ether (4 ml). The solid was collected, rinsed with ether and methanol, and dried in vacuo to give the pure product as a light yellow solid, 90 mg.

MS (APCI): 472 (M+1) for C₂₁H₁₉N₇O₄F₂

¹H NMR (DMSO-d₆) δ: 3.45-3.56 (m, 4H); 4.00 (m, 1H); 4.33 (t, 1H); 4.88-4.90 (m, 3H); 5.06 (t, 1H); 5.18 (m, 1H); 5.39 (s, 1H); 5.55 (s, 1H); 7.49 (dd, 1H); 7.67 (dd, 1H); 7.80 (t, 1H); 8.39 (d, 1H); 9.11 (s, 2H).

The intermediates for the above compound were prepared as follows:
3-(5-Bromopyrimidin-2-yl)-4,5-dihydroisoxazol-5-yl]methanol

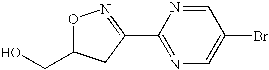

Methylmagnesium chloride (3M in tetrahydrofuran, 13.7 mL, 41.04 mmol) was added slowly to a solution of 2-iodo-5-bromo-pyrimidine (7.7 g, 27.4 mmol) in tetrahydrofuran (70 mL) at −78° C. The yellow solution was stirred for 30 min, then dimethylformamide (21.2 ml, 273 mmol) was added. The solution was slowly warmed up to room temperature and stirred for 2 hours. Methanol (50 ml) and water (50ml) were added follow by addition of hydroxylamine hydrochloride (3.77 g, 54.7 mmol) and sodium carbonate (1.74 g, 16.4 mmol) and the reaction mixture was allowed to stir for 1 hour. The suspension was poured into a mixture of dichloromethane (200 ml) and water (150 ml). The organic was separated, and the aqueous phase was washed with dichloromethane (2×200 ml). The organic phases were combined, dried over sodium sulfate, filtered, and concentrated in vacuo to give the oxime intermediate. This oxime intermediate was dissolved in tetrahydrofuran (100 ml), allyl alcohol (7.5 ml, 109 mmol) was added, follow by addition of bleach (195 ml, 136 mmol). The reaction mixture was allowed to stir for two hours at room temperature followed by extraction with tetrahydrofuran (2×250 ml). The organic phases were combined and concentrated. The residue was chromatographed on silica gel eluting with 50% hexane in ethyl acetate to give the title compound (1.5 g).

MS (APCI): 258 (M+1) for C₈H₈N₃O₂Br

NMR (DMSO-d₆) δ: 3.20-3.56 (m, 4H); 4.82 (m, 1H); 5.02 (m, 1H); 9.09 (s, 2H).
(5R)-5-{[4-(Fluoromethyl)-1H-1,2,3-triazol-1-yl]methyl}-3-[3-fluoro-4-(4,4,5,5-tetramethyl-1,3,2-dioxaborolan-2-yl)phenyl]-1,3-oxazolidin-2-one

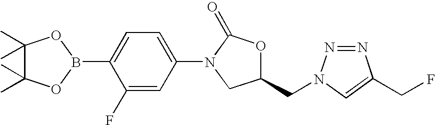

(5R)-3-(3-Fluoro-4-iodophenyl)-5-{[4-(fluoromethyl)-1H-1,2,3-triazol-1-yl]methyl}-1,3-oxazolidin-2-one (4.0 g, 9.5 mmol), bis(pinacolato)diboron (6.0 g, 23.75 mmol), potassium acetate (3.24 g, 33.25 mmol), and 1,1′-[bis(diphenylphosphino)ferrocene]dichloropalladium(II) dichoromethane complex (0.695 g, 0.95 mmol) were suspended in DMSO (25 ml). The mixture was heated at 80° C. for 90 minutes to give a clear black solution. After cooling down to room temperature, ethyl acetate (250 ml) was then added and the mixture was filtered through celite, washed with saturated NaCl (2×100 ml), dried over sodium sulfate and concentrated to dryness. The dark residue was dissolved in dichloromethane (30 ml), followed by slow addition of hexanes (100 ml), the resulting precipitate was filtered and washed with 5% dichloromethane in hexanes and collected as the desired product (2.73 g) which was used directly as an intermediate without further purification.

The intermediates for this example were prepared as follows:
(5R)-3-(3-Fluoro-4-iodophenyl)-5-[(4-fluoromethyl-1H-1,2,3-triazol-1-yl)methyl]oxazolidin-2-one

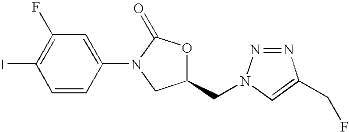

(5R)-3-(3-Fluoroiodophenyl)-5-[(4-bromomethyl-1H-1,2,3-triazol-1-yl)methyl]oxazolidin-2-one (6.94 g, 14.4 mmol) was dissolved/suspended in acetonitrile (250 mL) and water (1.5 mL). Potassium fluoride (4.19 g, 72.1 mmol) was added, followed by addition of 1-butyl-3-methylimidazolium tetrafluoroborate (18.4 mL) and the solution was heated to 90° C. over night. It was diluted with ethyl acetate, washed with water and dried over magnesium sulfate. Chromatography on silica gel with ethyl acetate gave 2.7 g (45%) of the title compound as an off-white amorphous solid.

MS (ESP): 421.34 (MH⁺) for C₁₃H₁₁F₂IN₄O₂

¹H-NMR (DMSO-d₆) δ: 3.88 (dd, 1H); 4.23 (dd, 1H); 4.84 (m, 2H); 5.14 (m, 1H); 5.45 (d, 2H, J_{H,F} 52 Hz); 7.14 (m, 1H); 7.49 (m, 1H); 7.81 (m, 1H); 8.34 (d, 1H).
(5R)-3-(3-Fluoro-4-iodophenyl)-5-[(4-bromomethyl-1H-1,2,3-triazol-1-yl)methyl]oxazolidin-2-one

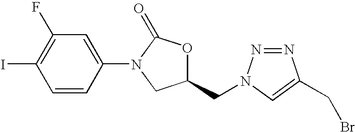

5R)-3-(3-Fluoro-4-iodophenyl)-5-[(4-hydroxymethyl-1H-1,2,3-triazol-1-yl)methyl]oxazolidin-2-one (14.7 g, 35.1 mmol) was suspended in dichloromethane (1 L). Carbon tetra bromide (12.16 g, 36.7 mmol) was added, it was cooled to 0° C. and triphenylphosphine (12.34 g, 61.2 mmol) was added. The mixture was stirred for 30 minutes at 0° C. and then at room temperature over night. For workup the reaction mixture was applied onto a silica gel column and eluted with hexanes/ethyl acetate (1:1) and then with ethyl acetate/methanol (95:5). Fractions containing product were pooled and recrystallized from ethyl acetate to give 14 g of the title compound as a colorless solid.

MS (ESP): 482.69 (MH⁺ for Br⁸¹) for C₁₃H₁₁BrFIN₄O₂

¹H-NMR (DMSO-d₆) δ: 3.87 (dd, 1H); 4.23 (dd, 1H); 4.74 (s, 2H); 4.81 (m, 2H); 5.12 (m, 1H); 7.14 (m, 1H); 7.49 (m, 1H); 7.81 (m, 1H); 8.22 (d, 1H).
(5R)-3-(3-Fluoro-4-iodophenyl)-5-[(4-hydroxymethyl-1H-1,2,3-triazol-1-yl)methyl]oxazolidin-2-one

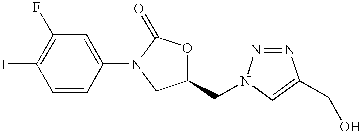

(5R)-3-(3-Fluoro-4-iodophenyl)-5-(azidomethyl)oxazolidin-2-one (10 g, 28 mmol) was dissolved in acetonitrile (80 mL). Propargyl alcohol (3.2 mL, 56 mmol) was added and then CuI (526 mg, 2.8 mmol) and it was stirred overnight. The solidified reaction mixture was extracted with ethyl acetate/acetonitrile, washed with water and dried over magnesium sulfate. Evaporation of solvent under vacuum gave 12.3 g crude product (quantitative).

MS (ESP): 419.13 (MH⁺) for C₁₃H₁₂FIN₄O₃

¹H-NMR (DMSO-d₆) δ: 3.88 (dd, 1H); 4.23 (dd, 1H); 4.51 (d, 2H); 4.80 (m, 2H); 5.14 (m, 1H); 5.22 (dd, 1H); 7.16 (m, 1H); 7.51 (m, 1H); 7.83 (m, 1H); 8.01 (d, 1H).
(5R)-3-(3-Fluoro-4-iodophenyl)-5-(azidomethyl)oxazolidin-2-one 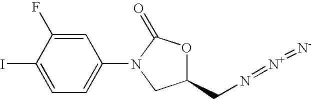
See International Patent Application, WO 03/035648 A1

1. A compound of the formula (I), or a pharmaceutically-acceptable salt, or an in-vivo-hydrolysable ester thereof, 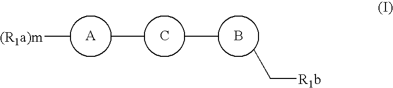
wherein in (I) C is a biaryl group C′-C″ 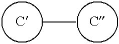
wherein C″ is an heteroaryl- or aryl-group selected from benzen-1,4-diyl, thien-2,5-diyl, and pyrid-2,5-diyl as shown in C″-1 to C″-3 below 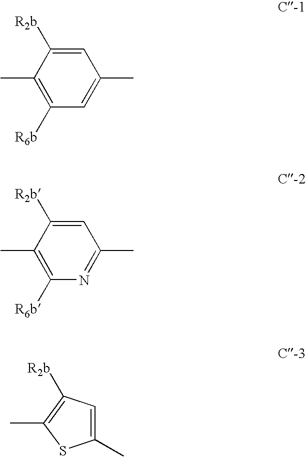
and C′ is an heteroaryl-group selected from pyridazin-3,6-diyl, pyrazin-2,5-diyl, pyrimidin-2,5-diyl (in either orientation), 1,3,4-thiadiazol-2,5-diyl, thiazol-2,5-diyl (in either orientation), and thiazol-2,4-diyl (in either orientation) as shown in C′-1 to C′-9 below: 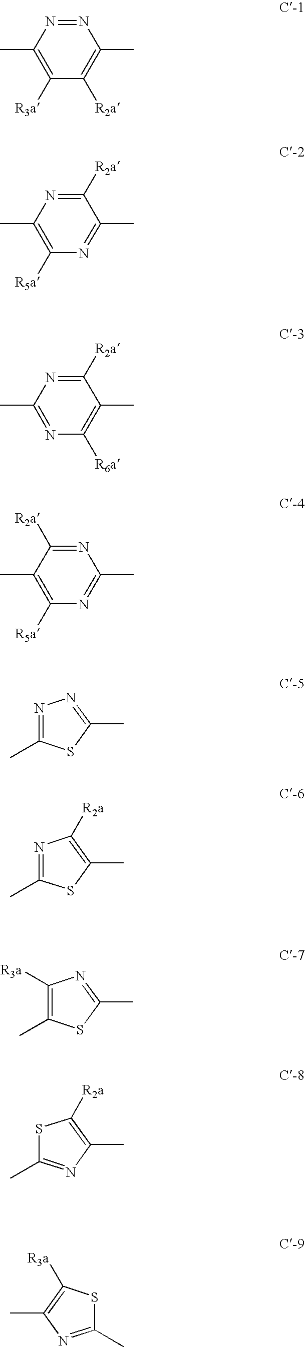
such that the central fragment C is represented by any one of the groups D to AD below: 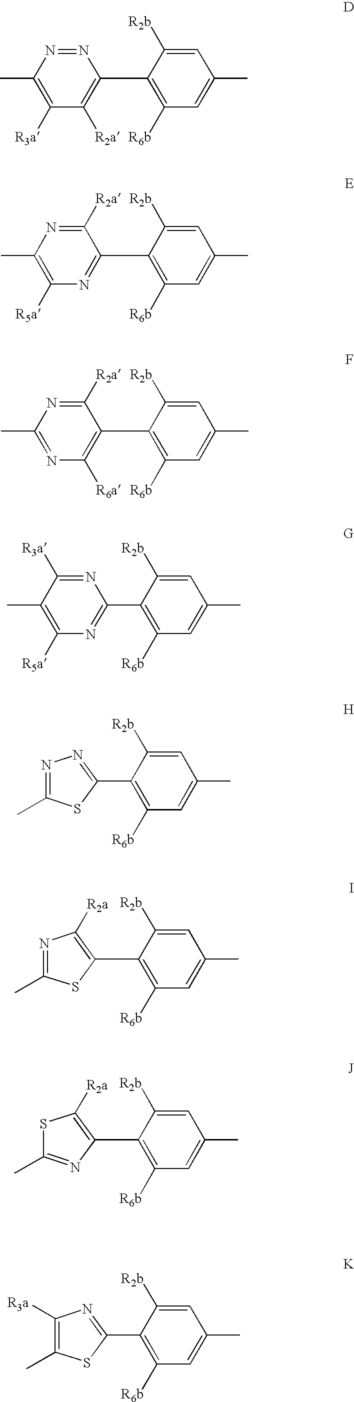 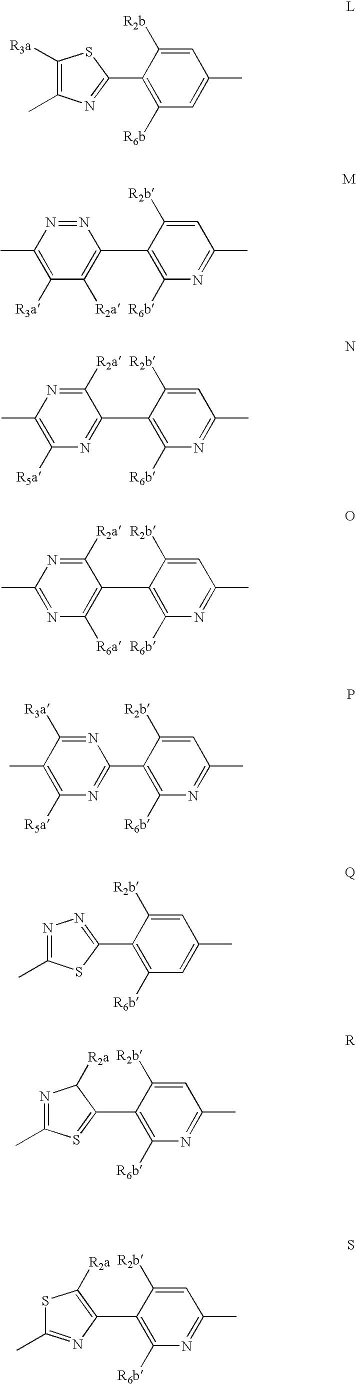 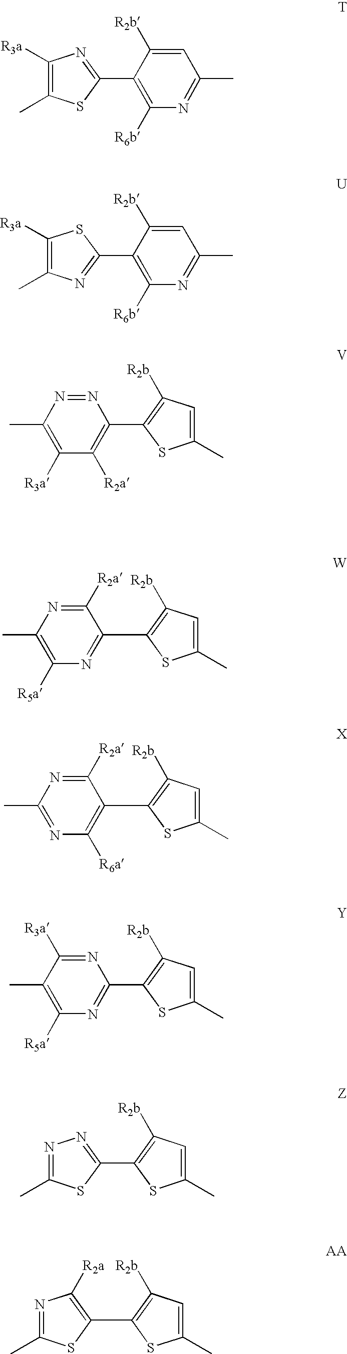 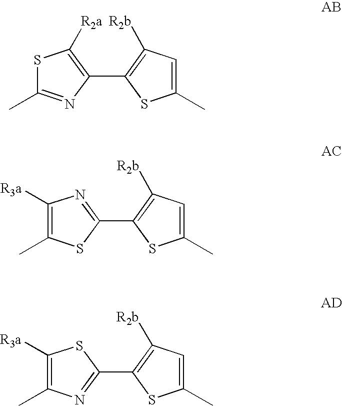
wherein the groups D to AD are attached to rings A and B in the orientation shown [(A-C′) and (C″-B)];
wherein A and B are independently selected from 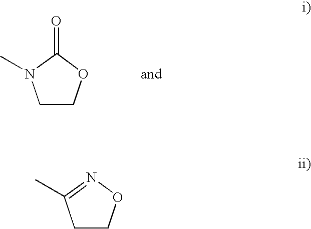
wherein A is linked as shown in (I) via the 3-position to ring C′ of group C and independently substituted in the 4 and 5 positions as shown in (I) by one or more substituents —(R₁a)m;
and wherein B is linked as shown in (I) via the 3-position to ring C″ of group C and independently substituted in the 5 position as shown in (I) by substituent —CH₂—R₁b;
R₂b and R₆b are independently selected from H, F, Cl, OMe, Me, Et and CF₃;
R₂b′ and R₆b′ are independently selected from H, OMe, Me, Et and CF₃;
R₂a is independently selected from H, Br, F, Cl, OMe, SMe; Me, Et and CF₃;
R₂a′ and R₆a′ are independently selected from H, OMe, SMe; Me, Et and CF₃;
R₃a is independently selected from H, (1-4C)alkyl, Br, F, Cl, OH, (1-4C)alkoxy,
—S(O)_{N}(1-4C)alkyl (wherein n=0,1, or 2), amino, (1-4C)alkylcarbonylamino-, nitro, cyano, —CHO, —CO(1-4C) alkyl, —CONH₂ and —CONH(1-4C)alkyl;
R₃a′ and R₅a′ are independently selected from H, (1-4C)alkyl, OH, (1-4C)alkoxy, (1-4C)alkylthio, amino, (1-4C)alkylcarbonylamino-, nitro, cyano, —CHO, —CO(1-4C)alkyl, —CONH₂ and —CONH(1-4C)alkyl;
wherein one of R₃a, R₃a′, R₅a′ taken together with a substituent R₁a at position 4 of ring A and rings A and C′ may form a 5-7 membered ring;
wherein any (1-4C)alkyl group may be optionally substituted with F, OH, (1-4C)alkoxy, —S(O)ₙ(1-4C)alkyl (wherein n=0,1, or 2) or cyano;
wherein when ring C′ is a diazine ring (D, E, F, G, M, N, O, P, V, W, X, Y) one of the ring nitrogens may optionally be oxidised to an N-oxide;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1 4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₁a3: (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N—(1-4C)alkyl-N-(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring;
wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1,
—CS(1-4C)alkyl and —C(═S)O(1-4C)alkyl; (═NORv) wherein Rv is as hereinbefore defined, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1O—, AR2—O—, AR3—O—, AR1—S(O)_{q}—, AR2—S(O)_{q}—, AR3—S(O)_{q}—, AR1—NH—, AR2—NH—, AR3—NH— (p is 1 or 2 and q is 0, 1 or 2), and also AR2a, AR2b, AR3a and AR3b versions of AR2 and AR3 containing groups, and additionally (1-6C)alkanoyloxy(1-4C)alkoxy, carboxy(1-4C)alkoxy, halo(1-4C)alkoxy, dihalo(1-4C)alkoxy, trihalo(1-4C)alkoxy, morpholino-ethoxy, (N′-methyl)piperazino-ethoxy, 2-, 3-, or 4-pyridyl(1-6C)alkoxy, N-methyl(imidazo-2 or 3-yl)(1-4C)alkoxy, imidazo-1-yl(1-6C)alkoxy}; wherein any (1-4C)alkyl, (1-4C)alkanoyl and (3-6C)cycloalkyl group present in any substituent on R₁a3 may itself be substituted by one or two groups selected from cyano, hydroxy, halo, amino, (1-4C)alkylamino and di(1-4C)alkylamino, provided that such a substituent is not on a carbon adjacent to a heteroatom if present;
R₁a4: R₁₄C(O)O(1-6C)alkyl wherein R₁₄ is AR1, AR2, AR2a, AR2b, (1-4C)alkylamino, or (1-10C)alkyl (optionally substituted as defined for (R₁a3), or alternatively R₁₄ is benzyloxy-(1-4C)alkyl, naphthylmethyl, (1-4C)alkoxy-(1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy, (1-4C)alkoxy-(1-4C)alkoxy-(1-4C)alkoxy, (1-4C)alkoxy-(1-4C)alkoxy, imidazo-1-yl(1-6C)alkyoxy(1-4C)alkyl, morpholino-ethoxy(1-4C)alkyl, (N′-methyl)piperazino-ethoxy(1-4C)alkyl, 2-, 3-, or 4-pyridyl(1-6C)alkyloxy(1-4C)alkyl, 2-, 3-, or 4-pyridyl(1-6C)alkylamino(1-4C)alkyl, 2-, 3-, or 4-pyridyl(1-6C)alkylsulfonyl(1-4C)alkyl, or N-methyl(imidazo-2 or 3-yl)(1-4C)alkyloxy(1-4C)alkyl;
R₁a5: F, Cl, hydroxy, mercapto, (1-4C)alkylS(O)p— (p=0, 1 or 2), —OSO₂(1-4C)alkyl, —NR₁₂R₁₃, —O(1-4C)alkanoyl, —OR₁a3;
m is 0, 1 or 2;
wherein two substituents R₁a at the 4 or 5 position of ring A taken together may form a 5 to 7 membered spiro ring;
wherein two substituents R₁a at the 4 and 5 positions of ring A taken together may form a 5 to 7 membered fused ring;
R₁b is independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 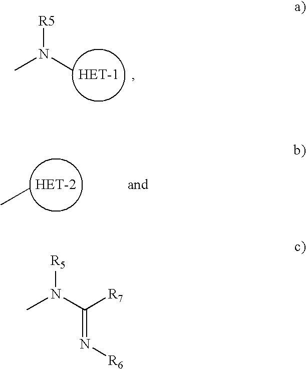
wherein W is O or S;
R₄ is hydrogen, amino, (1-8C)alkyl, —NHR₁₂, —N(R₁₂)(R₁₃), —OR₁₂ or —SR₁₂, (2-4C)alkenyl, —(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2, and additionally (2-6C)alkyl (substituted by 1, 2 or 3 substituents independently selected from methyl, chloro, bromo, fluoro, methoxy, methylthio, azido and cyano), and methyl (substituted by 1, 2 or 3 substituents independently selected from methyl, chloro, bromo, fluoro, methoxy, methylthio, hydroxy, benzyloxy, ethynyl, (1-4C)alkoxycarbonyl, azido and cyano);
R₅ is hydrogen, (3-6C)cycloalkyl, phenyloxycarbonyl, tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl; HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quaternised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro, and additionally (1-4C)alkoxycarbonyl; or
(RTa2) (1-4C)alkylamino, di-(1-4C)alkylamino, and (2-4C)alkenylamino; or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl; or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, —(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring); R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may additionally be taken together with the nitrogen atom to which they are attached to form an unsubstituted or substituted pyrrolidinyl, piperidinyl or morpholinyl ring, which ring may be optionally substituted by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl;
AR1 is an optionally substituted phenyl or optionally substituted naphthyl;
AR2 is an optionally substituted 5- or 6-membered, fully unsaturated (i.e. with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;
AR3 is an optionally substituted 8-, 9- or 10-membered, fully unsaturated (i.e. with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e. with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring;
wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by substituents selected independently from hydroxy, trifluoromethyl, (1-4C)alkyl S(O)q— (q is 0, 1 or 2), (1-4C)alkoxy, (1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)_{q}— (q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl; Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)_{q}— (q is 0, 1 or 2), (1-4C)alkoxy,
(1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

2. The compound of claim 1, wherein group C is represented by any one of groups D to L.

3. The compound of claim 1, wherein R₁a and R₁b are independently selected from —NHCO(1-4C)alkyl, —NHCO(1-4C)cycloalkyl, —NHCS(1-4C)alkyl, —N(R₅)-HET-1 and HET-2.

4. The compound of claim 1, wherein HET-2A is selected from the structures (Za) to (Zf) below: 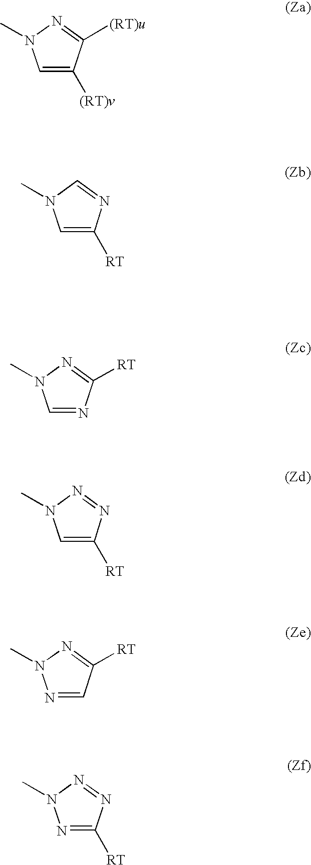
wherein u and v are independently 0 or 1.

5. The compound of claim 1, wherein RT is selected from
(a) hydrogen;
(b) halogen,;
(c) cyano;
(d) (1-4C)alkyl;
(e) fluoromethyl, choromethyl, bromomethyl, cyanomethyl, azidomethyl, hydroxymethyl;
(f) difluoromethyl, and trifluoromethyl.

6. The compound of claim 1, wherein at least one of A and B is an oxazolidinone.

7. The compound of claim 1, wherein both A and B are oxazolidinones.

8. The compound of claim 1, wherein group C is selected from groups F, H and I.

9. The compound of the formula (Ia) or a pharmaceutically-acceptable salt, or in-vivo hydrolysable ester thereof, wherein R₁a, A, C, B and R₁b are as stated in claim 1.

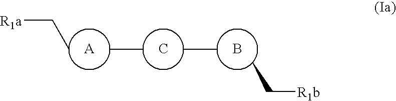

10. A pro-drug of a compound of claim 1.

11. A method for producing an antibacterial effect in a warm blooded animal which comprises administering to said animal an effective amount of a compound of claim 1, or a pharmaceutically-acceptable salt, or in-vivo hydrolysable ester thereof.

12. (canceled)

13. (canceled)

14. A pharmaceutical composition which comprises a compound of the invention as claimed in any one of claim 1, or a pharmaceutically-acceptable salt or an in-vivo hydrolysable ester thereof, and a pharmaceutically-acceptable diluent or carrier.

15. A process for the preparation of a compound of formula (I) as claimed in claim 1 or pharmaceutically acceptable salts or in-vivo hydrolysable esters thereof, which process comprises one of processes (a) to (h): and thereafter if necessary:
i) removing any protecting groups;
ii) forming a pro-drug (for example an in-vivo hydrolysable ester); and/or
iii) forming a pharmaceutically-acceptable salt;
wherein said processes (a) to (h) are:
(a) by modifying a substituent in, or introducing a substituent into another compound of the invention;
(b) by reaction of a molecule of a compound of formula (IIa) (wherein X is a leaving group useful in palladium coupling) with a molecule of a compound of formula (IIb) (wherein X′ is a leaving group useful in palladium coupling) wherein X and X′ are chosen such that an heteroaryl-aryl, or heteroaryl-heteroaryl bond replaces the heteroaryl-X and aryl-X′ (or heteroaryl-X′) bonds; 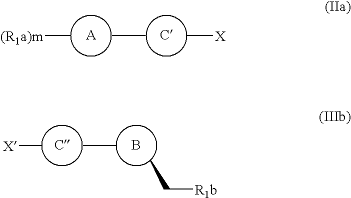
(c) by reaction of a (hetero)biaryl derivative (IIIa) or (IIIb) carbamate with an appropriately substituted oxirane (wherein 0, 1, or 2 of R₁a′-R₁a″″ are substitutents as defined for R₁a and the remainder are hydrogen) to form an oxazolidinone ring at the undeveloped aryl position; 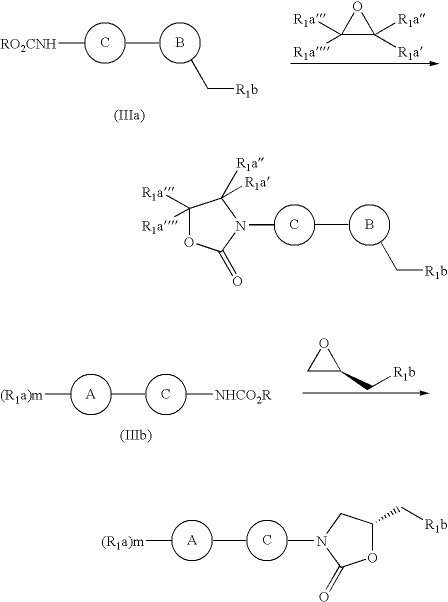
or by variations on this process in which the carbamate is replaced by an isocyanate or by an amine or/and in which the oxirane is replaced by an equivalent reagent X—C(R₁a′)(R₁a″)C(R₁a′″)(O-optionally protected)(R₁a″″) or X—CH₂CH(O-optionally protected)CH₂R₁b where X is a displaceable group;
(d) by reaction of a (hetero)biaryl derivative (IVa) or (IVb) to form an isoxazoline ring at the undeveloped aryl position; 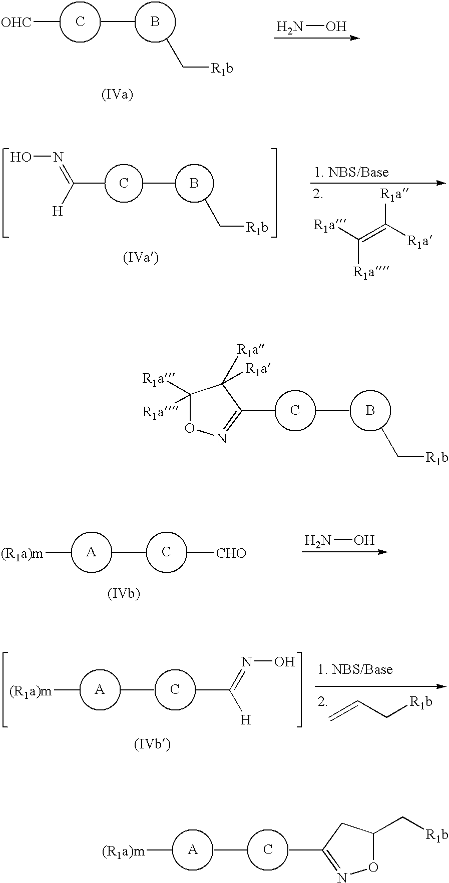
or by variations on this process in which the reactive intermediate (a nitrile oxide IVa″ or IVb″) is obtained other than by oxidation of an oxime (IVa′) or (IVb′); 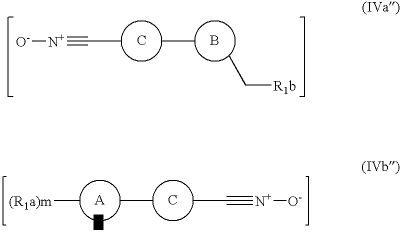
(e) for HET2 as optionally substituted 1,2,3-triazoles, by cycloaddition via the azide (wherein the substituent at the position of R₁a in (I) is azide) to acetylenes, or to acetylene equivalents such as optionally substituted cylcohexa-1,4-dienes or optionally substituted ethylenes bearing eliminatable substituents such as arylsulfonyl;
(f) for HET2 as 4-substituted 1,2,3-triazole, by reacting aminomethyloxazolidinones with 1,1-dihaloketone sulfonylhydrazones; 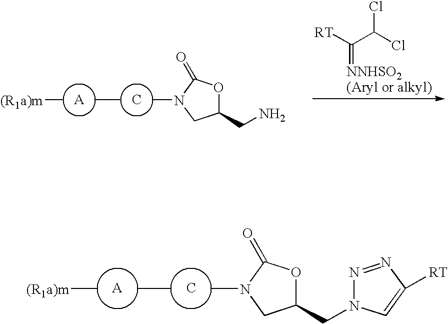
(g) for HET2 as 4-substituted 1,2,3-triazole, by reacting azidomethyl oxazolidinones with terminal alkynes using Cu(I) catalysis; 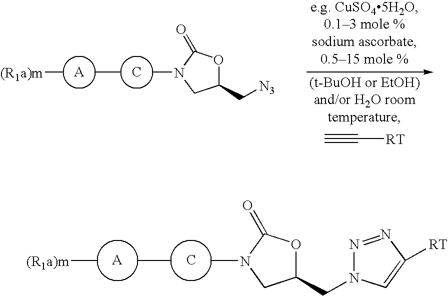
(h) for HET2 as 4-halogenated 1,2,3-triazole, by reacting azidomethyl oxazolidinones with halovinylsulfonyl chlorides at a temperature between 0° C. and 100° C. either neat or in an inert diluent. [chemical structure]
